(19) United States
(12) Patent Application Publication
(10) Pub. No.: US 2014/0320820 A1
(43) Pub. Date: Oct. 30, 2014

(54) METHOD AND DEVICE FOR MONITORING RETINOPATHY

(71) Applicants: Agency for Science, Technology and Research, Singapore (SG); Singapore Health Services Pte Ltd, Singapore (SG)

(72) Inventors: Saravana Kumar Kumarasamy, Singapore (SG); Tien Yin Wong, Singapore (SG); Barathi Veluchamy Amutha, Singapore (SG); Shyam Sunder Chaurasia, Singapore (SG)

(73) Assignees: Agency for Science, Technology and Research, Singapore (SG); Singapore Health Services Pte Ltd, Singapore (SG)

(21) Appl. No.: 14/266,491

(22) Filed: Apr. 30, 2014

Related U.S. Application Data
(63) Continuation-in-part of application No. 13/509,428, filed on May 11, 2012, now Pat. No. 8,833,942.

(30) Foreign Application Priority Data
Apr. 30, 2013 (SG) 201303340-2

(51) Int. Cl. A61B 3/10; A61B 3/00

(52) U.S. Cl. CPC A61B 3/10; A61B 3/0008
USPC 351/221; 351/246

(57) ABSTRACT

There is provided a method of monitoring retinopathy in a subject. The method involves measuring autofluorescence of a retina in response to high intensity blue light and infra-red reflectance of the anterior region of an eye in response to high intensity infra-red light of the subject over a total time period to obtain an autofluorescence intensity profile and an infra-red reflectance intensity profile. The autofluorescence intensity profile and infra-red reflectance intensity profile are processed to obtain a pupillary light reflex measurement that is used to assess the retinopathy status of the retina.

### CROSS-RELATED APPLICATIONS

This application is a continuation-in-part of U.S. patent application Ser. No. 13/509,428, which is a U.S. National Stage application under 35 U.S.C. §371 based on International Application No. PCT/SG2009/000422, filed Nov. 12, 2009, and claims the benefit of and priority from Singapore Patent Application SG 201303340-2, filed Apr. 30, 2013, the entire contents of each of which are incorporated herein by reference.

### FIELD OF THE INVENTION

The present invention relates to methods and devices for monitoring retinopathy, including retinal neuropathy and diabetic retinopathy.

### BACKGROUND OF THE INVENTION

Retinopathy is a non-inflammatory degenerative disease of the retina that leads to visual field loss or blindness. Retinopathy can be caused by various ophthalmic conditions as well as numerous systemic diseases outside the eye, for example diabetes. Diabetic retinopathy is an eye disease that results from damage to the retina as a result of complications such as nerve damage arising from diabetes mellitus. Diabetic retinopathy affects more than 80% of all patients who have had diabetes for 10 years or more and is the leading cause of vision loss in developed countries (Aiello et al., 1998).

Many retinal disorders can be diagnosed with the aid of retinal examination. Fluorescein angiography (FA) is the current standard technique used in diagnosis of diabetic retinopathy (DR) and is useful in detecting late-stage clinical hallmarks of DR, including retinal neovascularization (Saine, 1993). Laser photocoagulation, which has been applied in DR treatment for over half a century (Antonetti et al., 2006), is also a late-stage based treatment. Laser photocoagulation is successful in arresting proliferative diabetic retinopathy (PDR) in only 50% of cases. Even where further degeneration is prevented, any vision loss already incurred cannot be restored (Schwartz and Flynn, 2007).

Neuronal cell death in the retina (i.e. neuropathy) has been implicated in the early stages of DR, occurring much earlier than vascular damage becomes evident via FA techniques (Antonetti et al., 2006; Leith et al., 2000; Lorenzi et al, 2001; Gardner et al., 2002; Smith, 2007; Serrarbassa et al., 2008). Diabetic neuropathy affects the entire spectrum of retinal neurons, including the ganglion, horizontal, amacrine and photoreceptor cells (Antonetti et al., 2006; Smith, 2007). In fact, a reduction in the thickness of the neuronal cell layers in the retina due to diabetes has been reported in both experimental mice and human patients (Leith et al., 2000).

Pupillary light reflex (PLR) refers to the dilation/constriction of the pupil in response to light reaching the retina. High intensity light on the retina results in constriction in order to reduce the total light reaching the retina, and conversely, low intensity light results in pupil dilation in order to increase the light entering the eye and reaching the pupil. PLR can provide a useful diagnostic tool, allowing for testing of the sensory and motor responses of the eye. Lesions or disruptions in the eye can be detected by testing the direct response of a particular eye exposed to light entering the pupil as well as the consensual response of the eye when the opposite eye is exposed to light entering the pupil.

PLR has conventionally been used in the clinical setting to characterize the early effects of diabetic neuropathy (Hreidarsson, 1982; Devos et al., 1989; Kuroda et al., 1989). Such methods involve direct measurement of the pupil diameter or area in response to intense light. A pupillometer light source is generally focused on the pupil area, since the emphasis is on obtaining a bright and contrasting pupil image so that the pupil area can be accurately measured. This is an important consideration particularly in cases where poor pupil-iris contrast is obtained.

However, the early effects of retinopathy, which include diabetic neuropathy, on PLR can only be objectively assessed if light is directed onto the site of possible disease, that is, the photoreceptive retinal neurons. Thus, in order to use PLR as an indicator of early retinopathy, not only must the intensity level of the light source be controlled, but also the amount of light that reaches the retina must also be controlled (Fountas et al., 2006), which can be complicated, since slight changes in lens opacity and matrix of the eye may affect the amount of light actually reaching the retina. The initial pupil size may also affect the amount of light incident on the retina and influence the resultant pupillary constriction. As well, monitoring PLR by direct measurement of pupil size, such as pupil area or diameter, does not take into account the amount of stimulus light incident on the retina. Determining and standardizing the amount of light incident on the retina during diagnosis can present difficulties, particularly in longitudinal and quantitative studies of retinopathy.

Effective early detection and preventive treatment of retinopathy, including diabetic retinopathy would help to minimize complications such as permanent vision loss due to late-stage treatment provided by laser photocoagulation.

### SUMMARY OF THE INVENTION

The present invention relates to methods of monitoring retinopathy in a subject. Irradiation of the retinal ganglion cell (RGC) layer containing melanopsin-expressing retinal ganglion cells (mRGCs) with high intensity blue light results in constriction of the pupil. Measurement of PLR is typically used to assess early stages of retinopathy. The methods of the present invention use the autofluorescence (AF) of the RGC layer as an indicator of the level of PLR to monitor the retinopathy status of the retina.

The methods of the present invention are based on measuring the AF of the RGC layer containing mRGCs in response to high intensity blue light. An intensity profile of AF over time is obtained for a retina of a subject. The obtained profile is then processed in order to assess the retinopathy status of the retina.

The methods may also be performed in a dual mode, combining the AF excitation and measurement of the RGC layer using high intensity blue light together with infra-red (IR) imaging in order to obtain an image of the anterior region of the eye, thus allowing for direct measurement of the pupil and corneal sizes. This dual mode approach may provide an early detection method for use in individuals susceptible to developing retinopathy, including diabetic retinopathy.

In one aspect, the present invention provides a method of monitoring retinopathy in a subject, the method comprising: directing high intensity blue light at the retina of an eye of the subject; measuring autofluorescence of the retina in response to the blue light over a total time period to obtain an autofluorescence intensity profile; and processing the autofluorescence intensity profile to assess the retinopathy status of the retina.

The blue light may have a wavelength of from about 485 nm to about 490 nm, and in particular a wavelength of about 488 nm.

A confocal light source and/or a laser light source may be used to produce the blue light. In particular, a confocal scanning laser ophthalmoscope may be used in the methods of the invention.

The processing may include integrating area under the curve for the autofluorescence intensity profile.

The assessing may include comparing the processed autofluorescence intensity profile with a processed autofluorescence intensity profile obtained for retina of a non-diseased individual. Alternatively, the assessing may include comparing the processed autofluorescence intensity profile with a processed autofluorescence intensity profile obtained for the same retina of the subject.

In another aspect, the present method provides a diagnostic tool for monitoring retinopathy, the diagnostic tool comprising: a light source for generating high intensity blue light; a detector for detecting autofluorescence of a retina in response to the blue light; a memory, the memory storing instructions; and a processor in communication with the light source, the detector and the memory. The processor executes instructions to: activate the light source to generate the high intensity blue light directed at the retina of an eye of a subject; obtain an autofluorescence profile over a total time period from measurements at the detector; and process the autofluorescence intensity profile to assess the retinopathy status of the retina.

The diagnostic tool as described herein may be used in performing the method of the present invention.

In another aspect, the present invention provides a computer-readable medium storing executable instructions that, upon execution by a processor of a computing device, causes the computing device to facilitate monitoring of retinopathy by: generating high intensity blue light for directing at the retina of an eye of a subject; measuring autofluorescence of the retina in response to the blue light over a total time period to obtain an autofluorescence profile; and processing the autofluorescence intensity profile to assess the retinopathy status of the retina.

In yet another aspect, the present invention provides use of an autofluorescence intensity profile obtained for a retina in response to high intensity blue light over a total time period, for monitoring retinopathy in a subject.

In another aspect, the invention provides a method of monitoring retinopathy in a subject, the method comprising: directing pulses of high intensity blue light at the retina of an eye of the subject subject over a total time period; directing high intensity infra-red light at the anterior region of the eye of the subject over the total time period; measuring autofluorescence of the retina in response to the blue light to obtain an autofluorescence intensity profile; measuring infra-red reflectance of the anterior region of the eye in response to the infra-red light to obtain an infra-red reflectance intensity profile; processing the autofluorescence intensity profile and the infra-red reflectance profile to obtain a pupillary light reflex measurement in order to assess the retinopathy status of the retina.

The blue light may have a wavelength of from about 485 nm to about 490 nm, and in particular a wavelength of about 488 nm. The infra-red light may have a wavelength of from about 800 nm to about 490 nm, and in particular a wavelength of about 820 nm.

A confocal light source and/or a laser light source may be used to produce the blue light and the infra-red light. In particular, a confocal scanning laser ophthalmoscope may be used in the methods of the invention.

Processing may comprise identifying a constricted pupil area from the infra-red reflectance intensity profile and identifying a dilated pupil area from the infra-red reflectance profile. Processing may further comprise obtaining a lens autofluorescence measurement from the autofluorescence intensity profile.

Assessing may comprise comparing the processed pupillary light reflex measurement with a processed pupillary light reflex measurement obtained for an eye of a non-diseased individual or obtained for the same eye of the subject. Assessing may comprise comparing the processed lens autofluorescence measurement with a processed lens autofluorescence measurement obtained for an eye of a non-diseased individual or obtained for the same eye of the subject.

In another aspect, the invention provides a diagnostic tool for monitoring retinopathy, the diagnostic tool comprising: a light source for generating high intensity blue light and high intensity infra-red light; a detector for detecting autofluorescence of a retina in response to the blue light and infra-red reflectance of an anterior region of an eye in response to the infra-red light; a memory, said memory storing instructions; and a processor in communication with said light source, said detector and said memory, said processor executing instructions to: activate said light to generate pulses of said high intensity blue light directed at the retina of an eye of a subject; activate said light source to generate said high intensity infra-red light directed at the anterior region of the eye of the subject; obtain an autofluorescence profile over a total time period and an infra-red reflectance profile over the total time period from measurements at said detector; and process said autofluorescence intensity profile and said infra-red reflectance profile to obtain a pupillary light reflex measurement to assess the retinopathy status of the retina.

In yet another aspect, the invention provides a computer-readable medium storing executable instructions that, upon execution by a processor of a computing device, causes said computing device to facilitate monitoring of retinopathy by: generating pulses of high intensity blue light for directing at the retina of an eye of a subject and generating high intensity infra-red light for directing at the anterior region of the eye of the subject; measuring autofluorescence of the retina in response to the blue light over a total time period to obtain an autofluorescence profile; measuring infra-red reflectance of the anterior region of the eye in response to the infra-red light over a total time period to obtain an infra-red reflectance profile; and processing the autofluorescence intensity profile and the infra-red reflectance profile to obtain a pupillary light reflex measurement to assess the retinopathy status of the retina.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention,

FIG. 1 is a schematic representation of a diagnostic tool comprising a computing device that can facilitate performance of methods of the present invention;

FIG. 2 graphically illustrates variation in weight (A) and blood glucose levels (B) between FVB/N mice treated with saline (▪) or STZ (□);

FIG. 3 is in vivo time-lapse AF images of retina of saline and STX treated C57BL/6J mice;

FIG. 4 is representative AF intensity profiles for saline (A, B) and STZ (C, D) mice taken on day 0 (A, C) and day 28 (B, D);

FIG. 5 graphically illustrates variation in the mean area under the curve of saline (▪) and STZ (□) treated groups;

FIG. 6 shows micrograph images of morphologic abnormalities observed in mRGCs of retinas from control (A-C) and STZ-treated (D-F) mice;

FIG. 7 illustrates dual mode (both autofluorescence and infra-red) imaging via a confocal scanning laser ophthalmascope for stimulating and measuring pupillary light reflex in a subject;

FIG. 8 is a flowchart showing a method of pupil segmentation during light “ON” phase;

FIG. 9 illustrates pupil segmentation under autofluorescence mode;

FIG. 10 is a flowchart showing a method of iris segmentation during light “ON” and light “OFF” phases;

FIG. 11 illustrates iris segmentation from an infra-red image during both light “OFF” and “ON”;

FIG. 12 is a flowchart showing a method of pupil segmentation during light “OFF” phase;

FIG. 13 illustrates pupil segmentation during light “OFF”;

FIG. 14 illustrates segmentation of pupil and iris during light “OFF”;

FIG. 15 illustrates segmentation of a partially occluded pupil under autofluorescence mode;

FIG. 16 illustrates segmentation of pupil and iris under partial occlusion from eye lid and eyelashes during light “OFF” phase;

FIG. 17 illustrates measurement of lens autofluorescence using scanning laser ophthalmoscope;

FIG. 18 depicts a pupillary constriction profile for a mydriated eye under sustained exposure to blue laser light;

FIG. 19 is graphical representation of comparison between normalized pupil size/iris size (panel A) and absolute pupil size (panel B);

FIG. 20 is graphical representation of quantification of PLR measurements of various stages of diabetic retinopathy from mydriated eyes;

FIG. 21 is graphical representation of quantification of PLR measurements from non-mydriated eyes; and

FIG. 22 is graphical representation depicting the average ratio of pupil maxima to pupil minima from diabetic patients.

### DETAILED DESCRIPTION

In one aspect, the presently described method relates to indirect measurement of PLR by measuring retinal AF from a retina over a time course, due to stimulation of mRGCs with high intensity blue light.

The method measures the mRGC-mediated PLR response, since mRGCs are known to be intrinsically photosensitive (Lucas et al., 2001; Hattar et al., 2002; Tu et al., 2005; Wong et al., 2005; Schmidt and Kofuji, 2009). The mRGCs belong to a family of photosensitive neurons in the RGC layer that transduce light into electrical impulses which are then transmitted to the brain and processed. An appropriate response is then sent from the brain to the iris to regulate the size of the pupil.

The mRGCs play a complementary role with rod-cone photoreceptors in mediating PLR (Lucas et al., 2001; Hattar Et al., 2002; Tu et al., 2005; Belenky et al., 2003; Hattar et al., 2003; Lucas et al., 2003; Fu et al., 2005; Sekaran et al., 2005; Barnard et al., 2006; Guler et al., 2008; Hankins et al., 2008). The rod-cone system is active over a broad range of light intensities with peak sensitivity at 520 nm. In contrast, the mRGC-mediated pathway activates pupillary constriction at high light intensity levels (Lucas et al., 2003) and is most sensitive to blue light excitation, particularly at 488 nm (Lucas et al., 2001; Lucas et al., 2003; Grozdanic et al., 2007).

The AF obtained in response to the blue light is from AF aggregates within the RGC layer, for example lipofuscin pigments as well as advanced glycation end products, among others.

The method is based on obtaining an AF intensity profile of a retina over time. An AF intensity profile refers to the autofluorescence intensity measured in response to stimulation of the retina with the high intensity blue light over a given period of time, for example by taking successive time point measurements of AF intensity within a total time period, as described below.

The AF intensity profile obtained is processed and used to assess the retinopathy status of the retina. The AF intensity can be used as an indicator of the pupil size since the pupil regulates the amount of blue light that reaches the retina, which in turn regulates the AF that is emitted. The AF intensity profile obtained over time can be used as an indicator of the PLR, which in turn is an indicator of the health of retinal neurons. Thus, the AF intensity profile can be used to identify or monitor retinopathy.

This indirect measurement approach is a label-free imaging method that provides indirect assessment of the PLR of a retina, and may be used to monitor retinopathy, including retinal neuropathy and early stages of diabetic retinopathy, given the neuronal cell death associated with retinal neuropathy, including death of the mRGCs.

Thus, there is provided a method of monitoring retinopathy. The method comprises directing high intensity blue light at a retina in an eye of a subject in order to autofluoresce the retina; measuring the AF of the retina in response to the blue light over a total time period to obtain an AF intensity profile; processing the AF intensity profile in order to assess the retinopathy status of the retina.

In practising the method, high intensity blue light is directed at the retina of a subject in whom retinopathy is to be monitored, and in particular at the RGC layer, containing the mRGCs.

Blue light has a wavelength in the range of from about 450 to about 495 nm. Blue light is used in the method, as the mRGCs are most sensitive to blue light, particularly to light of wavelength 488 nm. In certain embodiments, the wavelength of the blue light is in the range of about 485 nm to about 490 nm. In a particular embodiment, the blue light has a wavelength of about 488 nm. In one embodiment, the blue light has a wavelength of 488 nm.

High intensity light is intended to refer to light that is of sufficient intensity to enable the collection of AF signal from the retina of the subject. Whether light is of sufficient intensity can be readily determined, for example by testing the intensity of light on a subject. If the light does not induce AF of the retina, despite being of an appropriate wavelength, then the light is not considered to be high intensity. It will be appreciated that the intensity of the light, while of high enough intensity to allow for measurement of the AF signal for the retina, will not be of such high intensity so as to damage the retina of the subject. For example, the intensity of the blue light may be chosen in keeping with standard limits, for example those established by the American National Standards Institute (ANSI Z136.1, 1993), or in keeping with international standards for safe use within a clinical setting.

The blue light may have an intensity of about 1 mW/cm² or greater, about 2 mW/cm² or greater, about 5 mW/cm² or greater, about 10 mW/cm² or greater, from about 1 mW/cm² to about 100 mW/cm², from about 1.0 mW/cm² to about 10 mW/cm², about 2 mW/cm², about 5 mW/cm² or about 10 mW/cm². For this range and all ranges given throughout this specification, any narrower range falling within a stated range is also intended.

The high intensity blue light is used to induce AF of the RGC layer in an eye of the subject. The retina is composed of several layers of neurons, including the RGC layer where the mRGCs are located. Thus, the blue light is directed on the retina of the subject, including the RGC layer, including the mRGCs in the retina.

The high intensity blue light may be generated at the appropriate wavelength, for example, using a scanning laser device, such as a confocal scanning laser ophthalmoscope. Use of the confocal scanning laser ophthalmoscope as the light source also allows for use as a photodetector for measuring the intensity of AF measured by the mRGCs.

A confocal or focused light source may be used, as light from such a source allows the light to be focused directly at the RGC layer within the retina. Use of a confocal light source, for example using confocal scanning laser technology, enables the light to be accurately directed, via optical sectioning, at the RGC layer where the mRGCs are located. It also enables continuous exposure of the RGC layer to high intensity blue light, including for times as long as half an hour, without causing any harm to the subject.

In order to ensure the blue light is directed at the RGC layer so that AF will be induced, the RGC layer may be located within the retina by first reflecting infra-red (IR) light to saturate the optic disc with the IR reflected brightness. For example, using a confocal scanning laser ophthalmoscope, the ophthalmoscope may be first operated in IR reflectance mode (for example, excitation: 820 nm, emission: all pass) such that the reflected IR brightness is saturated all around the optic disc (i.e. the corresponding pixels on the detector exhibit maximum intensity values). This provides a confirmation that the light is indeed focused onto the RGC layer. The ophthalmoscope may then be switched back to the fluorescence mode so that the blue laser light replaces the IR light. This approach enables the operator to control the intensity of blue laser light irradiating the RGC layer by first controlling the brightness of the reflected IR light prior to operating the ophthalmoscope in the fluorescence mode.

If other factors are present which affect the passage of light to the retina, such as changes to the lens opacity and matrix of the eye as well as initial pupil size, the method allows for tuning of the intensity of the blue laser light source to ensure that a given intensity of blue laser light actually reaches and irradiates the RGC layer. This may be done by first tuning the IR light source so that the reflected IR brightness is comparable to a reference IR brightness value. A calibration curve can then be used to determine the corresponding change required in the intensity of the blue laser light source so that the given intensity of blue laser light irradiates the RGC layer.

If desired, the subject's pupils may be chemically dilated prior to directing the high intensity blue light at the retina, in order to increase the amount of light that reaches the retina. Pupil dilation using chemical dilators such as mydriatics is known. Mydriatics are commercially available, for example cyclopentolate hydrochloride and tropicamide, including in formulations for administration as a drop to the eye.

In order to perform the method, the blue light is directed to the retina, including the RGC layer, for a total period of time over which the AF is to be measured and over which an intensity profile is to be generated. Thus, during the entire measurement period, the retina is exposed to the high intensity blue light.

The AF generated by the RGC layer in response to the high intensity blue light is measured over the total time period, using known methods for detecting fluorescence signals. A detector, such as a photo-detector, is used to detect the fluorescence signal generated by the retina. For example, laser confocal microscopy methods may be used, as described in U.S. Pat. No. 6,501,003, which may employ scanning laser ophthalmoscopy techniques. Such techniques may involve a scanning laser ophthalmoscope using a laser beam from a point source and then detecting reflected light using a photomultiplier. A confocal scanning laser ophthalmoscope provides AF images of high resolution, allowing for more precise quantification of the AF intensity and thus providing a more accurate AF intensity over time profile.

For example, fluorescent images detected using fluorescent microscopy techniques including ophthalmoscopy methods may be captured by computer and quantified using standard imaging software. Fluorescence images, including digital images, may be recorded using a photo-detector. The intensity of the AF recorded in a digital image may be determined by calculating pixel intensity within the image. For example, the average pixel intensity within a pre-determined area of an image may be calculated and used as the AF intensity for that image.

The light is directed to the retina and AF is measured over a total period of time. The total time period is a time period sufficient to measure the AF of the RGC layer and obtain an intensity versus time profile. The total time period may be, for example, 1 minute, 2 minutes, 5 minutes, or any time period falling within the range of from 30 seconds to 10 minutes. Sequential measurements of two or more AF measurements may be taken at time intervals over the total time period in order to generate a time course of measurements and obtain a profile of AF intensity over time. Measurements may be taken at any suitable time interval, for example: every 2 seconds, every 3 seconds, every 5 seconds, every 10 seconds or at any time interval falling within the range of from 2 to 20 seconds.

The AF measurements obtained may optionally be normalized with respect to an initial measurement (e.g. time=0) in order to produce relative AF values, which are thus expressed as a fraction, or relative amount of the initial AF measurement.

As indicated above, a series of intensity measurements are taken at time points throughout the total time period, resulting in a profile of AF intensity over time. If desired, the AF intensity profile may be expressed as a curve of intensity versus time, or relative intensity versus time.

The AF intensity profile is then processed in order to assess the retinopathy status of the retina for which the profile is obtained.

Processing includes any data manipulation or transformation applied to the data contained within the AF intensity profile. Processing may be performed on part or all of the profile. Processing may, for example, involve integration to obtain an area under the curve, taking a derivative of a portion or all of the profile, statistical analysis of the profile, averaging of the profile, application of filters, or image processing, for example to analyse morphological data contained within the digital images obtained during measurement.

In one embodiment, processing comprises integration of the AF intensity profile. The time course of AF measurements or relative AF is integrated over the total time period. An area under the curve is thus computed.

In addition to manipulation of the AF intensity profile, processing further includes using the processed intensity profile to assess the retinopathy state of the retina for which the intensity profile was obtained; that is, the processed intensity profile may be used to monitor the state of the neurons within the retina and the ability of the RGC layer to autofluoresce in response to high intensity blue light, and thus detect and/or monitor retinopathy within the eye. The processed intensity profile is thus used as an indirect assessment of the PLR of the particular retina tested, without any need to directly assess pupil diameter or area, allowing for assessment of the health of the retina and possible detection or monitoring even of early stages of retinopathy.

Retinopathy refers to any disease, disorder or condition which may cause, result in, or is associated with retinal degeneration including degeneration of the photoreceptors or mRGC neurons. The retinopathy may be any retinopathy, including primary retinopathy or secondary retinopathy, and includes, for example, degeneration of retinal neurons, neuropathy, glaucoma, retinitis pigmentosa and diabetic retinopathy. Retinopathy includes retinal gliosis, retinal degeneration or retinopathy related to neurodegenerative diseases including Parkinson's disease and Alzheimer's disease, primary retinopathies originating from the eye including retinoschisis, age-related macular degeneration and glaucoma, and secondary retinopathies originated from systemic diseases including diabetic retinopathy, hepatic retinopathy, renal retinopathy, hypertension, vascular diseases, congenital heart disease, autoimmune disorders including rheumatoid arthritis, multiple sclerosis, neurofibromatosis, Lyme neuroborreliosis, Down's syndrome, autism, sickle cell anaemia, infections with HIV and cytomegalovirus, thyroid disorders, or liver disorders.

The retinopathy status of a retina refers to the presence or absence of retinopathy or the extent of retinopathy or retinal degeneration in a particular subject at a particular point in time. Retinopathy status includes the stage of disease, including the stage prior to onset, as well as the extent of disease.

Retinopathy status may be assessed by comparing a processed AF intensity profile obtained for the retina of interest with a reference value. The reference value may be a value obtained from a processed AF intensity profile for an individual with a known retinopathy status, for example an individual known to have healthy, non-diseased retinas, or may be obtained for the same subject at an earlier point in time.

Thus, processing may include comparing the processed AF intensity profile for a particular retina with reference values obtained for an individual with a healthy, non-disease retina, in order to diagnose retinopathy or to monitor retinopathy progression within the eye of the subject. Thus, an individual that does not have retinopathy, or who is not at significant risk of developing retinopathy, or who does not have a known pre-disposition for developing retinopathy may be used to provide a standard of processed AF intensity profile in a healthy, non-disease retina, which standard is not indicative of or related to retinopathy, thus allowing for comparison of disease status in a subject that has, is suspected to have or that may be pre-disposed to develop retinopathy and an individual being free from any such pathology or pre-disposition. The value obtained for a particular retina may be compared with a reference value, in order to determine the retinopathy status of the retina and thus the eye. In this way, the processed intensity profile for the subject in the method may be used to diagnose the presence, onset or extent of retinopathy, including at stages earlier than typically possible using direct measurement of PLR.

Alternatively, processing may include comparing the processed AF intensity profile obtained in the method with a reference processed AF intensity profile value obtained at an earlier point in time for the same retina in the same subject, as a method of monitoring disease onset, disease progression, or disease regression in the subject. For example, comparison may be made between a current value and a value obtained from 1 to 4 weeks, from 1 to 9 months or from 1 to 5 years earlier for the same retina of the subject on which the method is currently performed.

It will be appreciated that in order to perform a relevant comparison, the method parameters used to obtain the reference value should be comparable to those used in the method for monitoring retinopathy. For example, using the same intensity and wavelength of blue light source and the same intensity of blue light irradiating the RGC layer, and comparing areas for the same total time period, and performing the same processing technique allows for comparison between retinas. As stated above, if factors such as the lens opacity, matrix of the eye and the initial pupil size for a given eye of a subject are such that the actual intensity of light irradiating the RGC layer is affected despite using a constant intensity of blue light from the light source, the intensity of the blue laser light source can be tuned so that the intensity of the blue light irradiating the RGC layer remains comparable with that used to obtain the reference value.

The subject on whom the method is performed is any subject for whom retinopathy is desired to be monitored. The subject may be any animal, including a mammal, including a human.

Monitoring retinopathy includes tracking disease onset, progression, regression, recovery or prognosis over a period of time and also includes tracking of response to treatment and tracking of side effects, including toxicity, of treatment, over a period of time. Thus, the monitoring may be performed during a treatment regimen for retinopathy by performing the method at various times throughout a time course of treatment or before, during or after treatment. Treatment may include dietary regimen, controlled environmental conditions, or administration of a therapeutic agent.

An assessment of the retina is a useful tool for determining the extent of an underlying disease in a non-invasive manner and may aid determination of prognosis and monitoring of disease progression in a patient. Due to its accessibility, examination of the retina may facilitate the assessment of therapeutic strategies and medical trials. Thus, the method described herein provides a non-invasive, label-free approach to quantitatively assess PLR of a retina in a subject.

The method is sensitive, since it relies on the AF from the RGC layer rather than measurement of the pupil itself, and therefore may allow for early detection of retinopathy, including retinal neuropathy and diabetic retinopathy, at a stage at which it may be possible to slow, prevent or reverse vision loss.

The method is more quantitative than the direct measurement of PLR since it can ensure a fixed amount of light reaches the RGC layer initially, allowing for a more reliable comparison between the processed AF intensity profiles of different subjects or at different time points in the same subject.

In some embodiments of the method, the method as described above may be adapted to combine the measurement of AF of the retina with IR reflectance imaging of the anterior region of the eye. This dual mode approach allows for the benefits and sensitivity provided by the blue light excitation of the retina in combination with direct measurement of the PLR using high intensity IR light. Such a dual mode approach may allow for early detection and possible treatment or prevention of retinopathy including diabetic retinopathy, for example in subjects that have been diagnosed with diabetes mellitus but have not yet shown signs of retinopathy onset.

Direct measurement of PLR can provide an effective tool for assessing the early effects of diabetes. However, conventional methods of measuring PLR can be inaccurate. When IR light is used alone in conventional methods, the intensity of the IR light is limited due to safety reasons; such limitation of light intensity in turn limits the pupil-iris contrast that can be achieved. Furthermore, sustained exposure to high intensity IR light may damage photoreceptors in the retina, which is particularly problematic given that photoreceptors do not have pain receptors directed to IR light.

Another drawback of conventional direct PLR measurement that may be addressed by the currently described methods is that pupil demarcation can be made difficult by occurrence of corneal reflectance, blinking, presence of eyelashes, head movement during measurement, etc. Corneal reflectance appears as white spots on the IR image of the pupil and can create interference if the pupil is constricted such that it partially overlaps with corneal reflectance spots.

Thus, combining the high intensity blue light AF measurement with high intensity IR reflectance measurement in the described methods allows for stimulation of both the mRGCs and the rod-cone photoreceptors in the eye, and allows for segmentation of the pupil area during light “ON” measurement phase in which the blue light is present and light “OFF” phase in which the blue light is absent. During the light “ON” phase, superior pupil-iris contrast may be achieved since the AF is only observed within the pupil region. This pupil-iris contrast then may be used to compensate for problems such as corneal reflectance and eyelash obstruction that can occur during the light “OFF” phase.

In addition, the AF images obtained during the light “ON” phase can provide a two-dimensional representation of lens autofluorescence (LA). LA has been implicated in various eye-related diseases including retinopathy such as diabetic retinopathy. Patients with higher LA read-out have a higher predisposition for developing diabetic retinopathy. Two-dimensional slices of AF data can be combined to generate three-dimensional volumetric data for LA.

Thus, the method described above for the single mode AF intensity profile may be modified to a dual mode method to obtain direct measurement of PLR. The manner in which the high intensity blue light is directed to the eye is modified, in combination with the use of high intensity infra-red light.

The following modifications may thus be made to the described single mode method of measuring AF intensity profile.

In contrast to the single mode AF described above, in which high intensity blue light is directed to the retina over the total period of time during which the AF is to be measured, in the dual mode, the method is adapted so that the high intensity blue light is directed to the retina, including the RGC layer, in pulses over the total period of time.

That is, over the total period of time, the high intensity blue light is pulsed in a binary mode, defined by “ON” and “OFF” phases. The “ON” phase refers to the period of the pulse during which the retina is exposed to the high intensity blue light. The “OFF” phase refers to the period of the pulse during which the retina is not exposed to the blue light.

The pulse cycles may be pulsed at a rate of from about 0.1 Hz to about about 5 Hz, or from about 0.2 Hz to about 3 Hz.

In some embodiments, the duration of the “ON” phase and the duration of the “OFF” phase may be of different length. In some embodiments, the duration of the “ON” phase and the duration of the “OFF” phase may be approximately of equal length. Thus, for example, the high intensity blue light may be pulsed on for from about 0.1 seconds to about 10 seconds and then turned off for from about 0.1 seconds to about 10 seconds with the “ON” and “OFF” phases of approximately equal duration, or may be pulsed on for about 5 seconds and then turned off for about 5 seconds.

In the dual mode approach, throughout the total time period, in addition to the pulsed blue light, high intensity IR light is continuously directed to the anterior region of the eye.

The anterior region of the eye is the front portion of the eye where light enters into the eye and includes the pupil and the iris. Thus, the IR light illuminates the anterior region, allowing for detection and measuring of the area of the pupil, which will dilate in the absence of the high intensity blue light.

Thus, when the blue light is “ON” and is being directed to the retina, the eye is exposed to high intensity IR light at the anterior region of the eye as well as the high intensity blue light directed at the retina. During the “OFF” phase of the pulse cycle, high intensity IR light continues to be directed to the anterior region of the eye, even though the retina is not exposed to blue light during the “OFF” phase.

As will be appreciated, IR light refers to light having a wavelength longer than visible light, for example in the range of 700 nm to 1 mm. Typically, near-IR light in the range of about 700 nm to about 1400 nm may be used, for example, IR light having a wavelength in the range of about 800 nm to about 850 nm, or IR light having a wavelength of about 820 nm.

As with high intensity blue light, high intensity IR light refers to light of sufficient intensity to enable the collection of data from the anterior region of the eye, including the pupil area. Whether the IR light is of sufficient intensity can be readily determined, for example by testing the intensity of light on a subject. As will be appreciated, pupil-iris contrast increases with increasing intensity of the IR light used. However, care should be taken to use an IR intensity necessary to achieve the desired result, but to avoid damaging the eye of the subject being assessed, in particular the retinal photoreceptors. IR light is used in existing methods of measuring PLR, and thus level of IR intensity and wavelength of IR light that can be used are known.

For example, the laser power of the IR light source used to generate the high intensity IR light may be about 5 μW to about 25 μW or about 10 μW to about μW at the objective lens.

The high intensity IR light may be generated using the same device as for the high intensity blue light, for example a scanning laser device, such as a confocal scanning laser ophthalmoscope, also fitted with an appropriate laser for emitting IR light. The use of the confocal scanning laser ophthalmoscope allows for the generation of both light wavelengths and provides a photodetector for measuring the AF of the retina as well as pupil area to determine PLR.

In addition to using IR light to first image the posterior region of the eye in order to locate the RGC layer as described above, a preliminary baseline image of the anterior region of the eye may also be obtained using IR reflectance. For example, using a confocal scanning laser ophthalmoscope, the ophthalmoscope may be operated in IR mode (excitation: 820 nm; emission: all pass) and directed at the anterior region of the eye by refocusing. The IR reflectance of the anterior eye may then be detected.

Thus, during the “ON” phase, the retina will be contacted with high intensity blue light and a constriction of the pupil is expected. During the “OFF” phase, the high intensity blue light will be removed and dilation of the pupil will be expected.

This response to the pulsing of blue light may be assessed by directly measuring changes in the pupil size from light “ON” to light “OFF” and vice versa, using the IR reflectance data. Notably, the average ratio obtained of pupil size during light “OFF” to pupil size during light “ON” for the total time period can be used as a measure of PLR.

Thus, the dual mode approach to this method allows for both measurement of the AF of the RGC layer to demarcate the pupil area, as well as the direct measurement of the PLR of the pupil in the anterior region of the eye.

As indicated above, the total time period for the pulse cycles is as described above for the single mode AF method. That is, the total time period is a time period sufficient to obtain sufficient pupil-iris contrast by autorfluorescing the retina during the “ON” phases and by directly measuring pupil area during the “OFF” phases of the total time period, in order to measure the PLR. The total time period may be, for example, 1 minute, 2 minutes, 3 minutes, 5 minutes, or any time period falling within the range of from 30 seconds to 10 minutes.

Accurate measurement of the pupil area and thus the PLR requires good pupil-iris contrast.

Thus, for each pulse that occurs during the total time period, there is an “ON” phase in which high intensity blue light is directed at the retina, including the RGC layer. The blue light will induce AF of the RGC layer; as a result, the pupil will emit the autofluorescence against a black background of the iris and the surrounding eye regions. The pupil-iris contrast is enhanced by the fact that the AF is limited to the pupil area, thus improving the demarcation between pupil and surrounding iris, as compared to conventional methods of measuring PLR. The use of the AF to define pupil area avoids problems associated with corneal reflectance of IR and occlusion by eyelashes.

Thus, as above in the single mode method, the AF profile may be measured over the total time period. In the dual mode approach, the AF profile is obtained for each “ON” phase occurring within the total time period. Sequential measurements of two or more AF measurements may be taken at time intervals within each “ON” phase over the total time period in order to generate a time course of measurements for each “ON” phase and obtain a combined profile of AF intensity over the total time. For example, in some embodiments, AF measurements may be taken every 0.1 second, every 0.2 second, every 0.5 second, every 1 second, every 2 seconds, every 3 seconds, every 5 seconds, every 10 seconds, every 20 seconds or at any time interval falling within the range of from 0.1 to 20 seconds.

Data obtained during the “ON” phase, can also be used to determine lens autofluorescence (LA). LA can be detected from within the pupil AF. The accuracy of PLR measurements obtained during the “OFF” phase rests on the assumption that a predefined amount of light reaches the retina. However, the actual amount of light that reaches the retina is dependent on the light transmission capability of the lens. LA provides an objective, accurate and reproducible method to quantify the transmission properties of lens. By doing so, the loss of light at the lens can be used to correct/normalize the PLR data from the “OFF” phase.

Thus, when a confocal scanning laser ophthalmoscope is used to perform the method, it is possible to obtain two-dimensional measures of LA, which can then be combined to generate three-dimensional volumetric LA measurement. This is in contrast to the use of conventional fluorometer-based methods of measuring LA, which only provide an average fluorescence vs. distance along optical axis. In the currently described methods, the collection of two-dimensional and three-dimensional LA data may allow for localized changes in a subject's LA to be tracked over time.

For each pulse that occurs during the total time period, there is also an “OFF” phase that occurs. IR light is directed to the anterior of the eye, during both “ON” phase and the “OFF” phase. When the high intensity blue light is switched off, the high intensity IR light continues to be directed to the anterior region of the eye, including the iris. As a result, during the “OFF” phase, since the pupil will not reflect IR light, the pupil will appear black against a white iris. When a confocal scanning laser ophthalmoscope is used, raster scanning and high intensity IR light can be used to achieve strong pupil-iris contrast.

Thus, an IR reflectance intensity profile may be measured over the total time period, including during each “ON” phase and each “OFF” phase occurring within the total time period. Repeated IR reflectance measurements may be taken at time intervals over the total time period, throughout the “ON” and “OFF” phases of each pulse cycle that occurs during the total time period. Measurements may be taken at any suitable time interval. For example, in some embodiments, IR reflectance measurements may be taken every 0.1 second, every 0.2 second, every 0.5 second, every 1 second, every 2 seconds, every 3 seconds, every 5 seconds, every 10 seconds, every 20 seconds or at any time interval falling within the range of from 0.1 to 20 seconds.

Thus, the AF intensity profile is obtained from the series of AF intensity measurements taken during the “ON” phases that occur during the total time period. Similarly, the IR reflectance intensity profile is obtained form the series of IR reflectance measurements taken during both the “ON” and “OFF” phases that occur during the total time period.

The AF intensity profile data collected during the “ON” phases and the IR reflectance intensity profile data collected during the “ON” and “OFF” phases, for example using a photodetector, including within a confocal scanning laser ophthalmoscope, can be used to determine the pupil area and the iris area during both the “ON” and “OFF” phases, thus allowing for direct measurement of the PLR.

Thus, together with the AF intensity profile, the IR reflectance intensity profile is also processed in order to obtain a processed PLR measurement, used to assess the retinopathy status of the retina of the eye for which the two profiles are obtained.

Various computer programs may be used for data extraction and analysis in order to determine the various states of the pupil and iris during the exposure to the various types of light. Mathematical and image processing routines, including ellipse fitting, binary morphology and segmentation can be used. Software, such as for example MATLAB software (Mathworks Inc., MA) may be used.

Processing may include determining pupil area directly from IR reflectance data obtained during both the “ON” phase and the “OFF” phase of the dual mode method.

That is, minimum pupil size (or pupil minima) of the constricted pupil can be determined for each “ON” phase that occurs during the total time period. Similarly, maximum pupil size (or pupil maxima) of the dilated pupil can be determined for each “OFF” phase that occurs during the total time period. The ratio of pupil maxima to pupil minima may then be calculated for each pulse cycle measured throughout the total time period. An average ratio may then be calculated, which may be used as the final PLR measurement for the eye.

Thus, processing may include segmenting the pupil area from the AF data collected during the “ON” phase. The AF image of the pupil may be contrast enhanced, for example using histogram equalization to improve signal-to-noise ratio. Grey scale “closing” and “opening” operations to suppress regions that are not part of the pupil area may be performed. For example, binary erosion and dilation operations using disc-shaped structuring elements may be performed to suppress background intensity. The segmentation may then be achieved via application of local intensity thresholds.

Processing may include using the “OFF” data collected during the period of the pulse cycle for which only the high intensity IR light is directed at the anterior region of the eye to determine the pupil area of the dilated eye, as the eye dilates upon removal of the blue light.

Processing may include pupil segmentation from the IR reflectance data using the partial iris region. If a Euler number of 0 exists, this indicates that a “hole” representing the pupil exists within the partial iris region. The pupil region may then be determined using ellipse-fitting routine on all boundary pixels. If a Euler number of 1 exists, the pupil region may then be segmented using an ellipse-fitting routine based on the valid pupil boundary pixels. Validity is determined by the distance between a boundary pixel at the outline of the partial iris region and the convex hull; the pupil arc does not contain any corners between end points.

Processing may include determining the iris region of the eye using IR reflectance data, which may be captured over the total time period, during both “ON” phases and “OFF” phases. For each IR image captured, the reflectance data of the anterior region of the eye may be processed as follows. The corneal reflectance within the pupil area is first segmented, followed by segmentation of a partial iris region. Valid pixels from the iris boundary are then identified based on distance from the corneal reflection. The complete iris region may then be reconstructed using an ellipse-fitting routine using the valid iris boundary pixels.

During processing, the pupil size may be normalized with respect to iris size, thus allowing for correction of artifacts that may be introduced due to head movement and tilt during measurement over the total time period.

Thus, it is possible to segment a partially occluded eye, for example, where the subject's eyelid or eyelashes cover part of the pupil or iris area during the exposure to the high intensity blue and IR lights during the total time period. The empirical selection of structuring elements of appropriate size and shape during segmentation contributes to the robustness of this approach. The binary erosion and dilation operations allow for reduction of background signal and for complete segmentation of the pupil even with occlusion by eyelashes.

The normalized pupil size and the use of corneal reflectance for pupil and iris segmentation, in the IR mode, also contribute to the robustness of the dual mode method. The use of a normalized pupil size may reduce inaccuracies due to subtle movements of a subject's head. For example, forward or backward tilt of a subject's head results in an apparent enlargement or reduction in pupil size as perceived by the optical detection system. Such artifacts may be removed with the normalization of pupil size with respect to iris size, as both pupil and iris would be subject to the same enlargement or reduction in size.

Processing may also include calculating a lens autofluorescence profile from the AF intensity profile. Lens autofluorescence data can be compiled to generate three-dimensional volumetric LA measurement.

Assessing retinopathy status of the retina may involve comparing the PLR measurement obtained with a reference PLR measurement. The reference measurement may be obtained from the processed AF intensity profile and IR reflectance profiles obtained for an individual having a known retinopathy status, for example an individual known to have healthy, non-diseased retinas, an individual with a known disease status, or may be obtained for the same subject at an earlier point in time thus monitoring disease onset and/or progression.

Thus, assessing may include comparing the processed PLR measurement for a particular retina with reference values obtained for an individual with a healthy, non-disease retina, in order to diagnose retinopathy or to monitor retinopathy progression within the eye of the subject. Thus, an individual that does not have retinopathy, or who is not at significant risk of developing retinopathy, or who does not have a known pre-disposition for developing retinopathy may be used to provide a standard of processed PLR measurement in a healthy, non-disease retina, which standard is not indicative of or related to retinopathy, thus allowing for comparison of disease status in a subject that has, is suspected to have or that may be pre-disposed to develop retinopathy and an individual being free from any such pathology or pre-disposition. The value obtained for a particular retina may be compared with a reference value, in order to determine the retinopathy status of the retina and thus the eye. In this way, the processed PLR measurement for the subject in the method may be used to diagnose the presence, onset or extent of retinopathy, including at stages earlier than typically possible using conventional methods of directly measuring PLR.

By comparing results obtained with this method for subjects with retinopathy and those without, it may be possible to assess disease state, and even to assess whether a patient is likely to develop retinopathy. For example, human subjects without diabetic retinopathy, even those having diabetes mellitus, tend to have, on average, higher initial baseline pupil constriction state than subjects that display early or moderate diabetic retinopathy. Upon exposure to blue light, the pupil constriction time for subjects with moderate diabetic retinopathy is, on average, longer than subjects without diabetes or subjects with diabetes who do not have diabetic retinopathy. Subjects with early or moderate DR also have, on average, poorer recovery than subjects without diabetes or subjects with diabetes who do not have diabetic retinopathy patients.

As indicated above, the dual mode approach also provides measurement of LA during the high intensity blue light “ON” phase. Measuring and monitoring of LA can provide an objective, accurate and reproducible method to quantify the transmission properties of lens, and may be monitored over time to assess disease progression. Multivariate analysis of LA data together with PLR data i.e. degree of constriction, re-dilation etc. with additional data such as gender, age, race can be used to obtain a score which is a scalar value predicting the likelihood of an early onset of various retinopathies, cataract, neurodegenerative diseases.

LA can also be used to longitudinally monitor the progression of cataract and to assess the efficacy of anti-cataract medications. Also, LA has been implicated in various eye-related diseases such as DR. Both PLR and LA are widely used as biomarkers in research laboratories and hospitals for monitoring early onset of eye related diseases such as retinopathy, macular degeneration and cataract as well as neurodegenerative diseases such as Alzheimer's and Parkinson's so that timely therapeutic intervention can be initiated.

Also provided are uses of high intensity blue light, including in combination with high intensity IR light, to measure PLR of an eye of a subject, including use of an AF intensity profile obtained for a retina in response to high intensity blue light over a total time period, and use of a PLR measurement obtained in response to high intensity blue light pulsed over a total time period and continuous high intensity IR light over the total time period for monitoring retinopathy in a subject.

The above-described methods and uses may be facilitated by a diagnostic tool comprising a computing device.

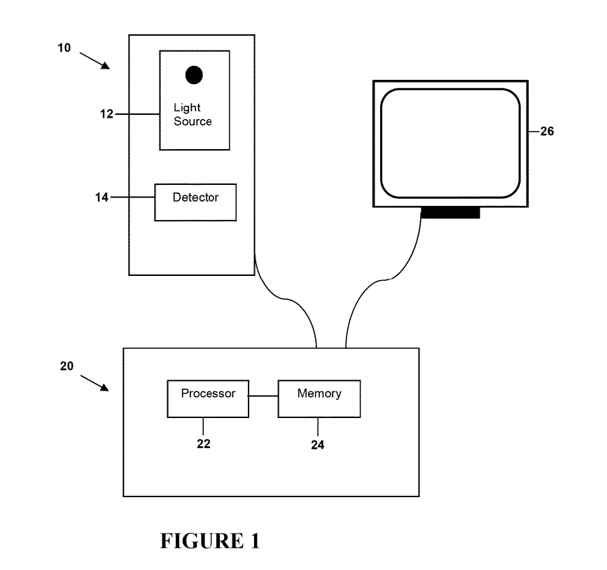

Referring to FIG. 1, the illustrated diagnostic tool 10 comprises a light source 12 for providing the high intensity blue light and high intensity IR light and a detector 14 for detecting AF from the retina in response to the blue light and for detecting and IR reflectance from the anterior region of the eye in response to the IR light.

Light source 12 produces the high intensity blue and IR light for the method as described above, including blue light having a wavelength in the range of about 485 nm to about 490 nm, or a wavelength of about 488 nm or a wavelength of 488 nm, and IR light having a wavelength in the range of about 800 nm to about 850 nm, or a wavelength of about 820 nm or a wavelength of 820 nm. Light source 12 may be, for example, a laser light source, a confocal light source, including a confocal laser. In a particular embodiment, light source 12 comprises one or more confocal scanning lasers.

In one embodiment, diagnostic tool 10 comprises a confocal scanning laser ophthalmoscope, which includes one or more confocal scanning lasers and a detector.

The diagnostic tool 10 further comprises, or is in communication with, a computing device 20 comprising a processor 22 and memory 24 in communication with the processor.

Processor 22 is typically a conventional central processing unit, and may for example be a microprocessor in the INTEL x86 family. Of course, processor 22 could be any other suitable processor known to those skilled in the art.

Memory 24 includes a suitable combination of random access memory, read-only-memory, and disk storage memory used by computing device 20 to store and execute software programs adapting diagnostic tool 10 to facilitate performance of the method.

Thus, computing device 20, including processor 22, is adapted to perform the method as described herein. For example, computing device 20 may control the wavelength and intensity of light generated by light source 12, and may communicate with detector 14 to receive and/or record the AF and IR reflectance data detected by detector 14. Computing device 20, and in particular processor 22, may be involved in executing the processing of the AF and IR reflectance data to produce a processed AF intensity profile and processed pupillary light reflex measurement, for example by transforming or manipulating data received from detector 14. Computing device 20 may also be involved in assessing the retinopathy status of a retina, for example by performing a comparison of a processed AF intensity profile or processed pupillary light reflex measurement with a reference value, such as a processed AF intensity profile or processed pupillary light reflex measurement obtained for a healthy, non-disease retina, or an earlier processed AF intensity profile or processed pupillary light reflex measurement, obtained at an earlier time point for a retina of the subject on which the method is performed.

The operation of the computing device and, in turn, the diagnostic tool, may be governed by software. The software, which takes the form of processor-executable instructions, may be loaded into the memory of the computing device from a machine-readable (e.g. computer-readable) medium, such as an optical disk or a magnetic storage medium for example.

The diagnostic tool may also further comprise, or may be in communication with, a display unit 26 for displaying the results of the method, for example, for displaying the measured AF or IR reflectance, for displaying the obtained AF or IR reflectance intensity profiles, for displaying the processed AF intensity profile or processed pupillary light reflex measurement, or for displaying the results of assessing the retinopathy status of the retina. For example, the display unit 26 may be in communication with the computing device 20, which in turn is in communication with the diagnostic tool 10 comprising the light source 12 and detector 14.

As well, computing device 20 may optionally include input/output devices, such as a keyboard, disk drive and a mouse (all not shown) or the like.

Thus, the diagnostic tool may be a confocal scanning laser ophthalmoscope incorporating or in communication with the computing device.

The present methods and uses are further exemplified by way of the following non-limited examples.

### EXAMPLES

### Example 1

The present study was designed to develop a novel method for quantifying PLR based on AF intensity (AFI) emitted due to confocal retinal blue light excitation (cRBLE) and to study longitudinal PLR alteration in a type 1 diabetic mouse model. Diabetes was triggered via a single intraperitoneal injection of streptozotocin (STZ) into wild type C57BL/6J mice. Anaesthethized mice were subjected to cRBLE weekly over a period of four weeks. At each time point, PLR was quantified by introducing the concept of the ‘area under the curve’ (AUC) of the intensity profile of retinal AF measured at 5 second (s) intervals over a period of 275 s. The mice develop diabetes as early as three days after STZ induction. The blood glucose levels peaked at approximately 23 mmol/L and the body weight decreased by approximately 20% after one month post-treatment. A progressive decrease in diabetic AUC occurred during this period but control AUC remained relatively unchanged. PLR was initiated despite synaptic blocking of the iris circular muscles during mydriasis followed by anaesthesia.

Research Design and Methods

Animal Husbandry.

10-week old male C57BL/6J mice were used for the present study. Animals were housed at the Biological Resource Center in a controlled environment (room temperature at 21° C. and a 12 h light/dark cycle) with free access to food and water in Biopolis, Singapore. The experimental protocol covering the current study was approved by the Institutional Animal Care and Use Committee (IACUC).

Induction of Diabetes.

Diabetes was induced using the streptozotocin (STZ) pharmacological model. A single dose of streptozotocin (STZ), dissolved in sodium citrate buffer (0.1M, pH4.5), was administered intra-peritoneally to the mice at 200 mg/kg body weight. The mice developed diabetes within three days after administration. In the STZ-treated group (n=4), a base-line cRBLE was performed prior to the STZ treatment, followed by STZ treatment the very next day. cRBLE was performed once weekly for a total of 4 weeks. Saline was used as a vehicle for the control group (n=5). Care and use of animals adhered to the institutional guidelines for humane treatment of animals.

Determination of Blood Glucose.

Mice were bled via tail-end puncture and the blood glucose levels were measured with a glucometer (AccuCheck; Roche Diagnostics Asia Pacific Pte. Ltd., Singapore). Mice with fasting blood glucose levels higher than 13.9 mmol/L were considered diabetic.

Preparation of animals. Mice were anaesthetized by intra-peritoneal (i.p.) injections with 0.15 ml/10 g body weight of Avertin (1.5% 2,2,2-tribromoethanol; T48402) purchased from Sigma-Aldrich (St. Louis, Mo., USA), and the pupils dilated with a drop of 0.5% Cyclogyl® sterile ophthalmic solution (cyclopentolate hydrochloride, Alcon®, Puurs, Belgium). Custom-made PMMA hard contact lenses (from Cantor & Nissel, Northamptonshire, UK) were used to avoid dehydration of the cornea and minimized spherical aberration of the mouse eye which could compromise the cRBLE procedure. Careful eye examination ruled out the presence of any corneal or lens opacities. The pupils were dilated for 15 minutes before the cRBLE procedure.

Confocal Retinal Blue Light Excitation (cRBLE).

A commercially available confocal scanning laser ophthalmoscope (cSLO), the Heidelberg Retina Angiograph 2, HRA 2 (Heidelberg Engineering, Dossenheim, Germany) (26; 27), was used for the cRBLE procedure on the mice. The 30° focal lens was replaced with a 55° wide angle objective lens in order to allow more light to enter the small mouse pupil. The 100% argon laser power provided maximum excitation intensity at the desired wavelength of 488 nm (and emission at ≧500 nm). The laser was consistently focused on the RGC layers since this is the focal section where the mRGCs are located. This focal section was located by first operating the cSLO in the infra-red (IR) reflectance mode (excitation: 820 nm, emission: all pass) and by ensuring that the resultant IR brightness was saturated all around the optic disc with the laser power and photo-detector sensitivity fixed at 50% and 65% respectively. The cSLO was then switched back to fluorescence mode before acquiring the images. Pupillary constriction was measured indirectly based on the amount of AF emitted from the focal section by a photo-detector fixed at 93% sensitivity. The AF detected decreases as the pupil constricts. A series of time-lapse AF images at 5 s intervals for a period of 275 s was acquired for each mouse eye. The OS (left) eye was first subjected to cRBLE, after which the mouse was adapted in the dark for 10 minutes before the procedure was repeated on the OD (right) eye.

Image-based PLR quantification. An intensity profile of AF for a period of 275 s was obtained by computing the average pixel intensity of the corresponding image at each 5 s interval. The area under the ‘intensity vs. time’ curve (AUC) is then computed and used here as a measure of PLR. The AUC for a particular mouse on any given day and eye is normalized with respect to its day 0 (base-line) AUC. The quantified AUC values for a specific time point and experimental group, i.e. saline or STZ treated, are expressed as A̅U̅C̅±SEM, where A̅U̅C̅ denotes the average AUC value across all mice at a specific time point and experimental group whereas SEM denotes the corresponding standard error of the mean. The results were statistically tested using the Student's two-tailed t-test assuming unequal sample variance.

Immunohistochemistry.

On day 28 post-STZ treatment, mice were killed by CO₂ asphyxiation in a gas chamber. The eyes were immediately enucleated and placed overnight in 4% paraformaldehyde (PFA) in phosphate buffered saline (PBS). The anterior part (cornea, lens, and vitreous) of the eye was removed and the retina was carefully isolated free of the pigment epithelium. The retinas were fixed in fresh 4% PFA in PBS for 30 minutes and then washed three times in PBS for 5 minutes each. The free-floating retinas were first blocked with 3% bovine serum albumin (BSA) for 1 hour at room temperature and were then incubated with a primary melanopsin antibody (polyclonal rabbit anti-melanopsin; Affinity Bioreagents, Golden, Colo.) at 1:200 dilution in PBS/0.3% Triton X-100/3% bovine serum albumin for 72 hours at 4° C. After three washes in PBS of 15 minutes each, the fluorescence-conjugated secondary antibody (Alexa Fluor 594 goat antibody to rabbit immunoglobulin G; Molecular Probes, Eugene, Oreg., USA) was applied to the sample as previously described, except that incubation was for 2 hours at 37° C. The retinas were washed again as described above, flat mounted onto glass slides and coverslips were applied using Vect Mount Permanent Mounting Medium. (Vector Laboratories, Burlingame, Calif., USA).

### FIGURE LEGENDS

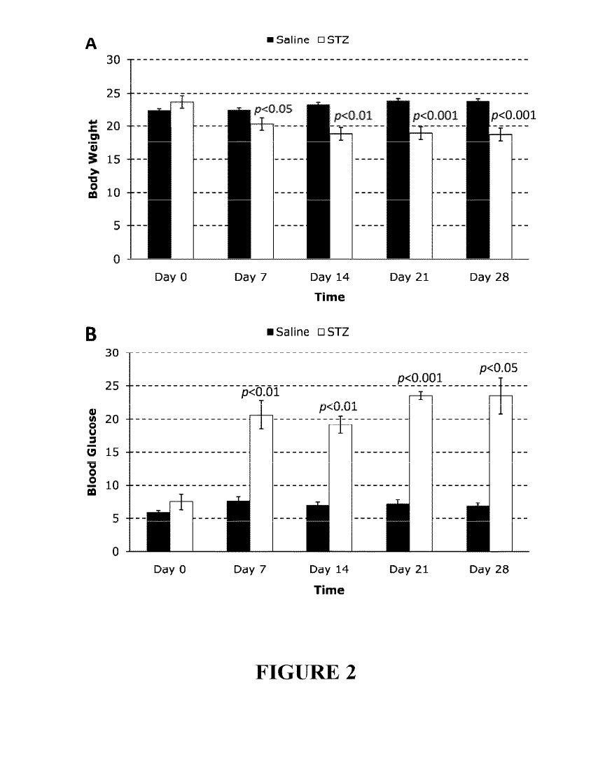

FIG. 2. Longitudinal variation in average weights and blood glucose levels of saline (n=5) and STZ treated (n=4) FVB/N mice. The difference in mean body weight (A) between the two groups is significant on days 7 (p<0.05), 14 (p<0.01), 21 (p<0.001) and 28 (p<0.001). The difference in mean blood glucose levels (B) is also significant on days 7 (p<0.01), 14 (p<0.01), 21 (p<0.001) and 28 (p<0.05).

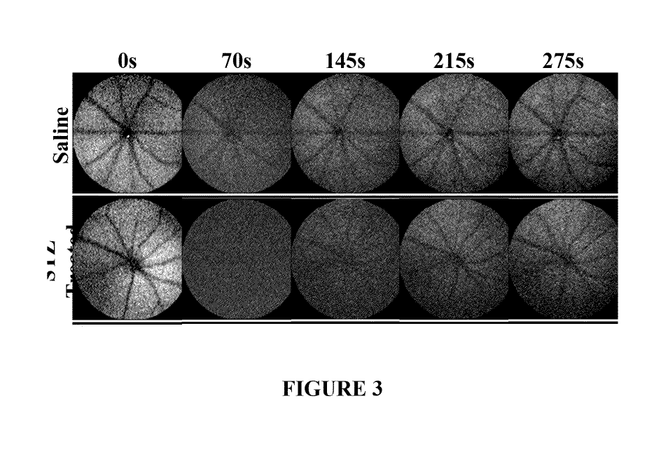

FIG. 3. In vivo time-lapse AF images of the saline and STZ treated wild type C57BL/6J mouse retina, centered around the optic disc, over a period of 275 seconds. The AFI is a measure of the mouse pupil size and both saline and STZ treated mice have comparable intensity levels at t=0 seconds. But subsequently the STZ treated mice show a more distinct fall in AFI compared to saline as shown at t=70 s. The STZ treated mice also show a slower recovery in AFI as shown from t=70-275 seconds. All images have been denoised and contrast-enhanced.

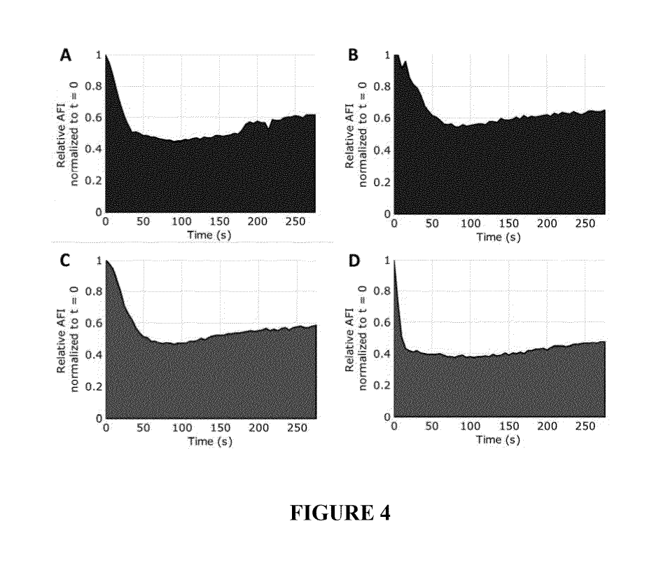

FIG. 4. Comparison of representative AFI profiles for saline on day 0 (A) and day 28 (B) and STZ treated mice on day 0 (C) and day 28 (D). Each AFI profile has a series of AFI values at 5 second intervals over a period of 275 seconds. Each AFI value has been normalized with respect to its t=0 value. The AUC for the saline and STZ treated mice are labelled blue (A, B) and red (C, D) respectively.

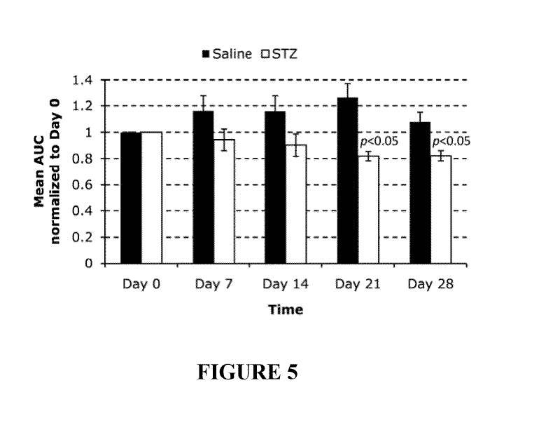

FIG. 5. Longitudinal variation in the mean AUC (AUC) of the saline (n=5) and the STZ treated (n=4) groups. The AUC measurements for day 0 were normalized to 1, as a reference for comparing the later longitudinal time points. The A̅U̅C̅ value of the STZ treated group drops steadily from days 0-28. But the saline group shows a higher A̅U̅C̅ from days 7-28 relative to its base-line value. The difference between the two groups is significant on days 21 (p<0.05) and 28 (p<0.05). The mean AUC values were expressed as A̅U̅C̅±SEM and statistically tested using the Student's two-tailed t-test assuming unequal sample variance.

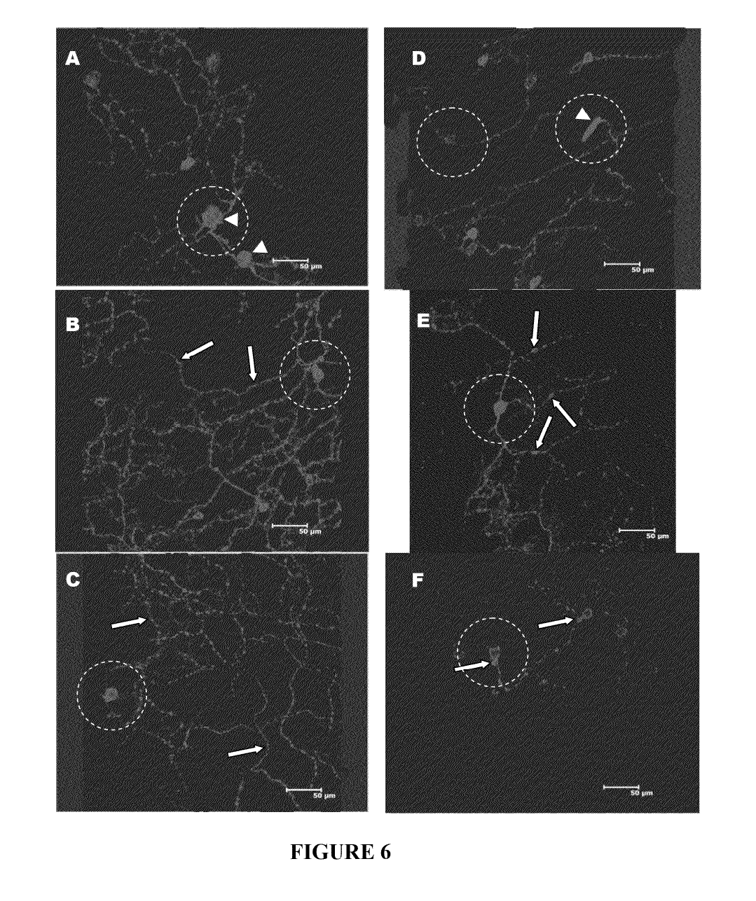

FIG. 6. Morphologic abnormalities in mRGCs of three different pairs of control (A-C) vs. STZ treated (D-F) mice. Flatmount mouse retinas were labeled with an antibody to melanopsin and imaged by confocal microscopy. A qualitative Sholl analysis (37) is made where a circle (dashed white line) of 50 μm radius is centered around some of the somas and the number of dendrites intersecting with the circle gives an indication of the extent of dendritic arborisation. There are fewer dendrites radiating from the soma in the STZ set compared to the control set. Some swelling is observed in the varicosities (see thin white arrows) along the dendrites of the STZ set whereas the control set appeared normal. Morphological difference is observed in the soma (see white arrow head) of the first pair (A, D) where the soma of the STZ mice appears elongated whereas that of the control mice is more spherical in shape.

Results

Blood Glucose and Body Weight Levels.

FIG. 2A shows the mean body weight of the STZ-treated mice falling gradually from day 0 to day 28 relative to the saline group where the difference between the two groups becomes significant (p<0.05) from day 7 onwards. FIG. 2B shows a sharp rise in the mean blood glucose level of the STZ treated mice, relative to the saline, where the difference in mean blood glucose levels becomes significant (p<0.01) from day 7 onwards. The STZ treated mice exhibit symptoms of polydipsia and polyuria from day 7 onwards.

In Vivo Imaging of Retinal AF in Diabetic Mice.

FIG. 3 shows a representative pair of selected time-lapse AF retinal images acquired from the RGC layer over 275 s from day 28 saline and STZ treated C57BL/6J mice. Both pupils exhibit PLR characterized by an initial decrease in AF (pupillary constriction) followed by a gradual increase in AF (light adaptation). But the STZ case shows a faster initial constriction and slower light adaptation when compared to the saline case. This trend is also reflected in the corresponding AF intensity profile of both pupils in FIGS. 3C and D with respect to the corresponding base-line profiles in FIGS. 3A and B. The AUC is computed from the shaded regions under the curve.

Data Quantification and Statistical Analysis.

The AUC was quantified for each eye of every mouse belonging to the saline (n=5) and STZ treated (n=4) groups and the data is presented in FIG. 5. The AUC value of the STZ treated group drops steadily from days 0-28. The saline group shows a higher AUC from days 7-28 relative to its base-line value. The difference between the two groups is significant on days 21 (p<0.05) and 28 (p<0.05).

Melanopsin Immunoreactivity.

FIG. 6 shows a number of morphological differences between three control (FIG. 6A-C) and three STZ treated (FIG. 6D-F) mice with regards to the axons and somas of mRGCs. Firstly, the STZ treated group shows far fewer number of primary dendrites around the soma than the control group, indicative of severe atrophy. Secondly, swelling is observed in the varicosities along the dendrites of this group (see thin white arrows). Lastly, morphological differences are also observed in the somas, where the somas of the STZ group appear elongated in the first pair (A and D) or condensed in the third pair (C and F), both reminiscent of apoptotic cells, when compared to the more spherical somas in the control group.

The data from FIG. 6 suggests that the rapid constriction in the STZ treated group followed by the delayed pupillary dilation may be due to morphological and functional changes undergone in the mRGCs, although the exact mechanism is still unknown. Similar changes in the morphology of melanopsin expressing cells were also reported by Gastinger et al. in the Ins2^{Akita/+} diabetic mouse model, where a link between these cells and the secondary effects of diabetes in the eye, i.e., diabetic retinopathy, was shown. The present data show that these effects can be observed within four weeks of diabetes. In fact, Martin et al. have reported that retinal neurons in the GCL of STZ treated mice undergo apoptosis as early as two weeks after the onset of diabetes. Therefore, PLR under the excitation of high intensity blue light can be an effective in vivo physiological marker for studying the early effects of diabetic retinopathy.

### Example 2

In this study, the method involves the use of (i) a scanning laser ophthalmoscope (cSLO) with a reflectance IR and a 488 nm blue laser in dual-mode operation and (ii) an image processing software for the automated quantification of pupil size. A total of 9 subjects volunteered for this study (3: negative control, 1: diabetic but no DR, 1: early DR and 4: moderate DR). Confocal scanning laser ophthalmoscopy was used to induce and to record PLR. The experiments were conducted in the morning where the left eye, and subsequently right eye, was subjected to 488 nm blue light excitation for 3 minutes, during which time changes in pupil size were monitored.

Research Design and Methods

Patient Recruitment.

Patients were recruited at SNEC Diabetic Retinopathy Services (DRS). A total of 9 subjects volunteered for this pilot study (3: negative control, 1: diabetic but no DR, 1: early DR and 4: moderate DR).

PLR Acquisition.

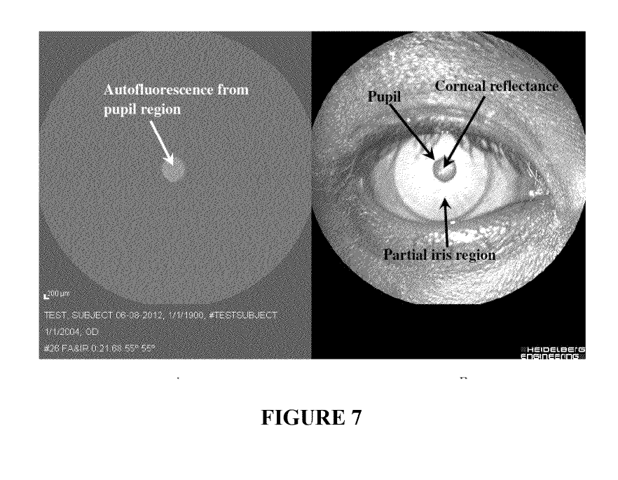

PLR measurements were taken in the morning at 10 am for all subjects. The subject is first dark adapted for approximately 15 minutes after which the patients' retina is imaged under infra-red (IR) reflectance mode (exc.: 820 nm, em.: all pass) to (i) ascertain the degree of occlusion in the optical medium between the anterior and posterior eye and (ii) to localize the retinal area for this study. A commercially available confocal scanning laser ophthalmoscope (cSLO), the Heidelberg Retina Angiograph 2, HRA 2 (Heidelberg Engineering, Dossenheim, Germany) was employed for the retinal imaging procedure. A base-line anterior image of the eye is first acquired in IR mode before operating the device in dual-mode where blue laser light (480 nm exc.) is pulsed into the eye, at the localized retinal region with a frequency of 0.2 Hz, while the pupil image is captured under IR mode at a fixed time interval over a total period of 3 minutes. The infra-red mode enables visualization of the anterior eye and segmentation of the iris and pupil (when blue light is switched off) for the purpose of quantifying pupillary light reflex. FIG. 7 shows the simultaneous dual mode (autofluorescence and infra-red) images acquired via a confocal scanning laser ophthalmoscope (cSLO).

Pupil Segmentation During Light “ON”.

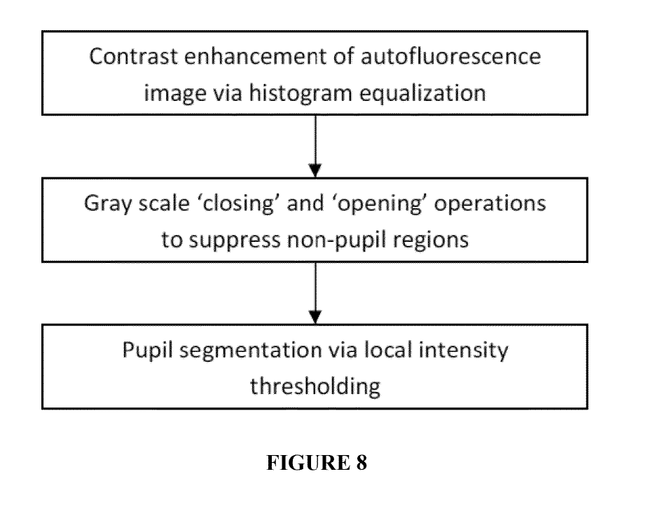

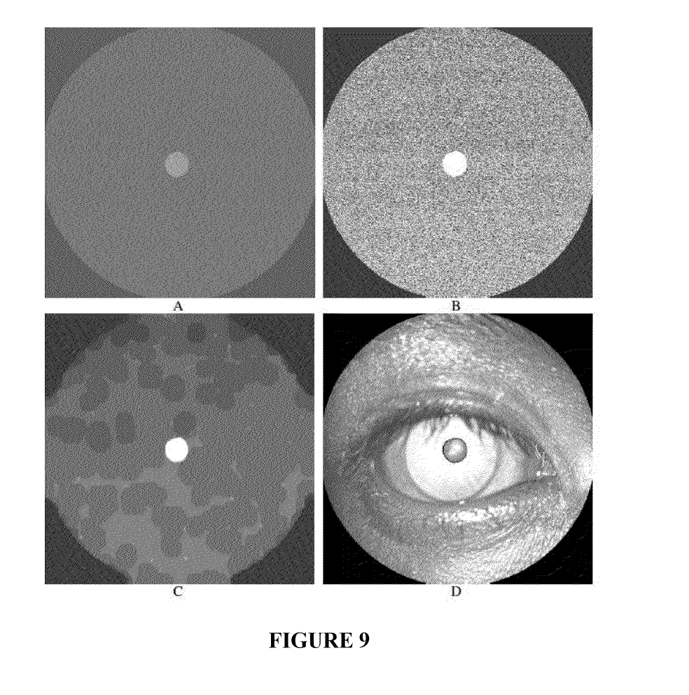

The crystalline lens is the most highly autofluorescing component in the eye. Since the pupil provides a clear unimpeded access to the lens, lens autofluorescence is detected from within the entire pupil. The significantly higher pupil autofluorescence compared to background (iris and cornea) autofluorescence enables accurate pupil segmentation. The overall methodology is as follows: FIG. 8 shows the flowchart for pupil segmentation during light “ON” whereas FIG. 9 illustrates the corresponding steps involved.

Iris Segmentation from the IR Image During Light “OFF” and “ON”.

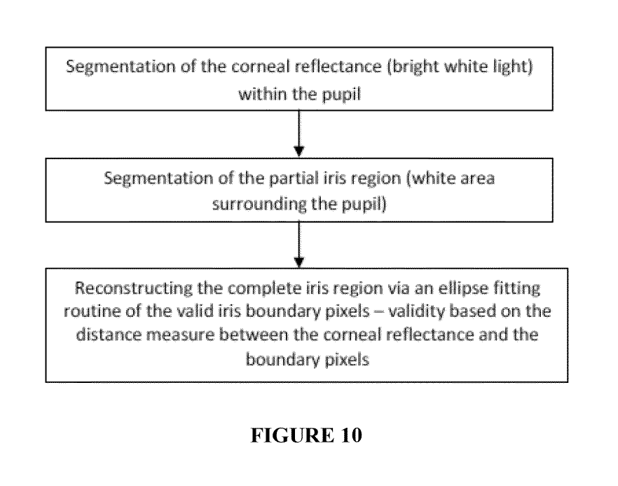

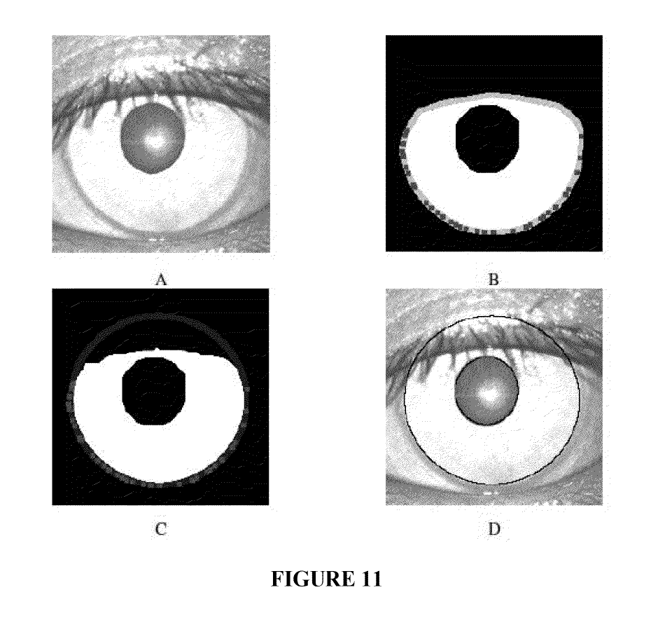

FIG. 10 shows the flowchart for iris segmentation during both the light “ON” and light “OFF” phases whereas FIG. 11 provides a more detailed illustration of the steps involved. FIG. 5 shows the three sequential steps in segmenting the iris from the anterior eye (i) segmentation of the corneal reflectance (bright white light) within the pupil—FIG. 11A (ii) segmentation of the partial iris region (white area surrounding the pupil)—FIG. 11B (iii) determining valid pixels from the iris boundary based on the distance measure from the corneal reflectance—FIG. 11C (iv) reconstructing the complete iris region via an ellipse fitting routine of the valid iris boundary pixels—FIG. 11D.

Pupil Segmentation During Light “OFF”.

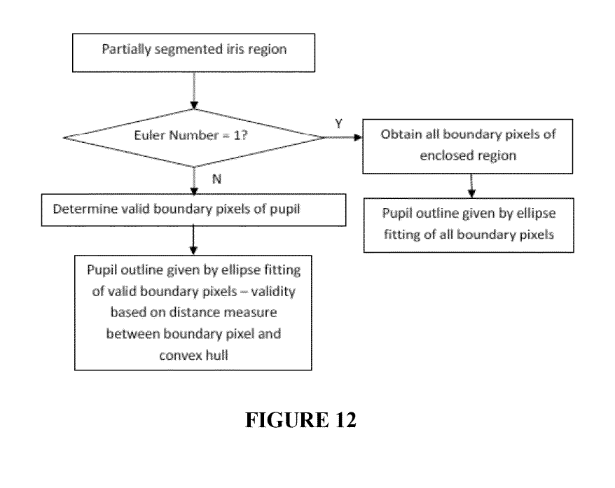

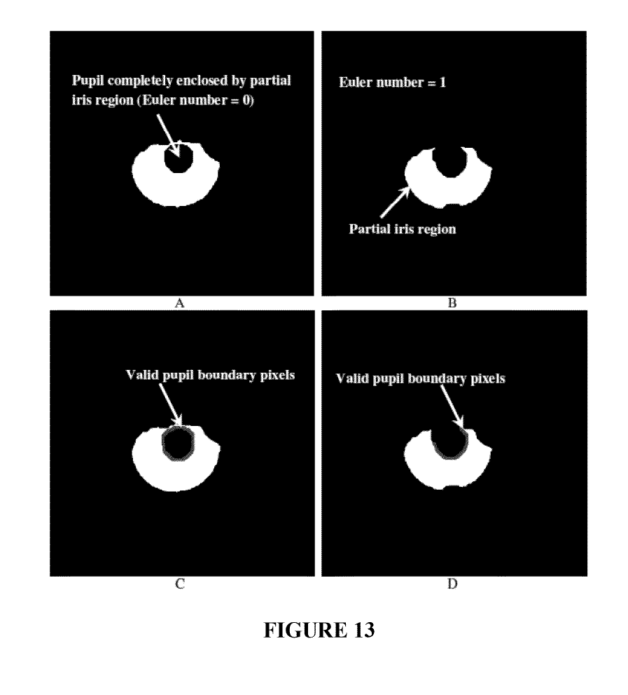

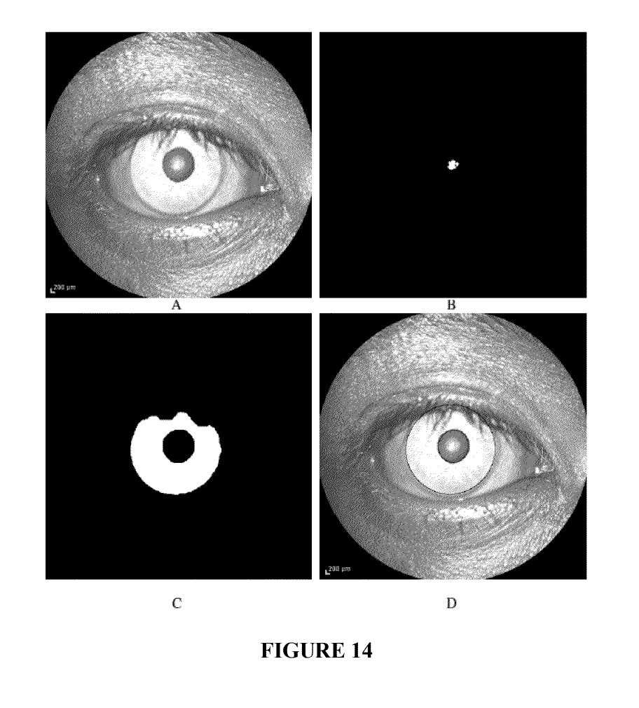

The pupil is segmented from the IR image during light ‘OFF’. An Euler number of 0 for the partial iris region indicates that a ‘hole’ (connected black pixels) exists within the partial iris region (connected white pixels) and this ‘hole’ represents the pupil region. Alternatively, if the Euler number is 1, the pupil region is segmented via an ellipse fitting routine of the valid pupil boundary pixels. The pupil boundary pixels lie on the outline of the partial iris region away from the convex hull AND the pupil arc does not contain any corners between its terminal end points. FIG. 12 shows the flowchart for pupil segmentation during light “OFF” whereas FIG. 13 provides a detailed illustration of the steps involved. FIG. 14 provides a further illustration of both pupil and iris segmentation during light “OFF”.

PLR Measurement of Mydriated Eyes.

A total of 9 subjects volunteered for this study (3: negative control, 1: diabetic but no DR, 1: early DR and 4: moderate DR). Confocal scanning laser ophthalmoscopy was used to induce and to record PLR. The experiments were conducted in the morning where the subjects' eyes were dilated with tropicamide and dark adapted for 45 minutes. The left eye, and subsequently right eye, was subjected to 488 nm blue light excitation for 3 minutes, during which time changes in pupil size were monitored.

PLR Measurement of Non-Mydriated Eyes.

A total of 2 subjects volunteered for this study (1: negative control, 1: diabetic but no DR). Confocal scanning laser ophthalmoscopy was used to induce and to record PLR. The experiments were conducted in the morning where the patients were dark adapted for 15 minutes. The left eye, and subsequently right eye, was subjected to pulses of 488 nm blue light excitation for 3 minutes, during which time changes in pupil size were monitored.

Both iris and pupil sizes are quantified at fixed time intervals throughout the 3 minute pulsing of light. For every pulsing cycle (once every five seconds or 0.2 Hz), the degree of pupillary constriction and constriction time during light ‘ON’ and degree of pupillary redilation and redilation time during light ‘OFF’ are recorded. Over a 3 minute period, a sufficiently large number of data sets (≈18) are available so that statistical significance of parameter differences, if any, between non-DR and DR patients can be more reliably ascertained.

Data Extraction and Analysis.

Pupil and iris segmentation required several mathematical and image processing routines such as ellipse fitting, binary morphology and segmentation, which were executed in the MATLAB software (Mathworks Inc., Natick, Mass.) with the aid of the Image Processing Toolbox.

### FIGURE LEGENDS

FIG. 7. Simultaneous dual mode (autofluorescence and infra-red) imaging via a confocal scanning laser ophthalmoscope (cSLO) for stimulating and measuring pupillary light reflex. A. Blue laser light (480 nm) is directed into the subject's pupil and the corresponding lens autofluorescence is detected A 55 o wide angle objective lens was used to enable an increased retinal field of view and to allow more light through the pupil for increased autofluorescence read-out. The 100% argon laser power ensures maximum excitation intensity at the desired wavelength of 488 nm (and emission at _{—} 500 nm) The lens autofluorescence is used here for segmenting the pupil during blue light excitation B. Infra-red mode of the same field of view as in A. The IR laser power and photo-detector sensitivity are fixed at 25% and 85% respectively. A base-line anterior image of the eye is first acquired in IR mode before operating the device in dual-mode where blue laser light is pulsed into the eye, at the localized retinal region with a frequency of 0.2 Hz, while the pupil image is captured under IR mode at a fixed time interval over a total period of 3 minutes. The infra-red mode enables visualization of the anterior eye and segmentation of the iris and pupil (when blue light is switched off) for the purpose of quantifying pupillary light reflex.

FIG. 8. Flowchart for pupil segmentation during light “ON”.

FIG. 9. Pupil segmentation under autofluorescence (AF) mode. A Image of the anterior eye under AF mode B Image A subject to histogram equalization. Histogram equalization enhances the contrast between the pupil and the background noise C Image B subject to binary erosion and dilation operations using disc-shaped structuring elements for suppressing the background intensity levels. D Pupil segmentation as indicated by the outline in black.

FIG. 10. Flowchart for iris segmentation during both the light “ON” and light “OFF” phases.

FIG. 11. Iris segmentation from the IR image during light ‘OFF’ and ‘ON’. The iris in image (A) is segmented via three sequential steps: (i) segmentation of the corneal reflectance (bright white light) within the pupil; (ii) segmentation of the partial iris region (white area surrounding the pupil demarcated by the light gray line in B); (iii) determining valid light gray pixels from the iris boundary based on the distance measure from the corneal reflectance; (B) (iv) reconstructing the complete iris region via an ellipse fitting routine of the valid iris boundary pixels (C and D).

FIG. 12. Flowchart for pupil segmentation during light “OFF” phase.

FIG. 13. Pupil segmentation during light ‘OFF’. The pupil is segmented from the IR image during light ‘OFF’. An Euler number of 0 (A) for the partial iris region indicates that a ‘hole’ (connected black pixels) exists within the partial iris region (connected white pixels) and this ‘hole’ represents the pupil region (C). Alternatively, if the Euler number is 1 (B), the pupil region is segmented via an ellipse fitting routine of the valid pupil boundary pixels (D). The pupil boundary pixels lie on the outline of the partial iris region away from the convex hull AND the pupil arc does not contain any corners between its terminal end points.

FIG. 14. Segmentation of pupil and iris during light “OFF” phase. (A) Image of the anterior eye under light “OFF” IR mode (B) Segmented corneal reflectance region from image A (C) Segmented partial iris region from image A (D) Segmented results of the complete pupil and iris regions as indicated by the black outline.

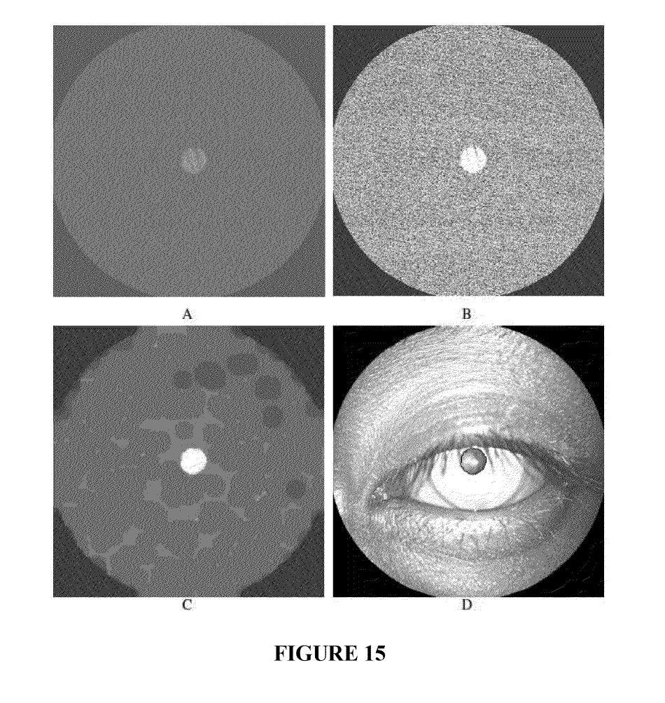

FIG. 15. Segmentation of partially occluded pupil under autofluorescence (AF) mode. A: Image of the anterior eye under AF mode. B: Image A subject to histogram equalization. Histogram equalization enhances the contrast between the pupil and the background noise. C: Image B subject to binary erosion and dilation operations using disc-shaped structuring elements for suppressing the background intensity levels. D: Pupil segmentation as indicated by the outline in black.

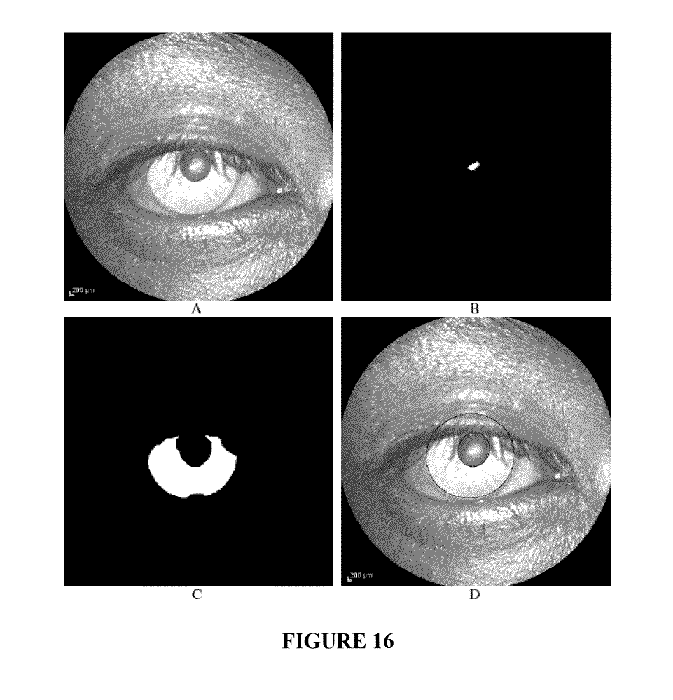

FIG. 16. Segmentation of pupil and iris under partial occlusion from eye lid and eyelashes during light “OFF” phase. (A) Image of the anterior eye under light “OFF” IR mode; (B) Segmented corneal reflectance region from image A; (C) Segmented partial iris region from image A; (D) Segmented results of the complete pupil and iris regions as indicated by the black outline

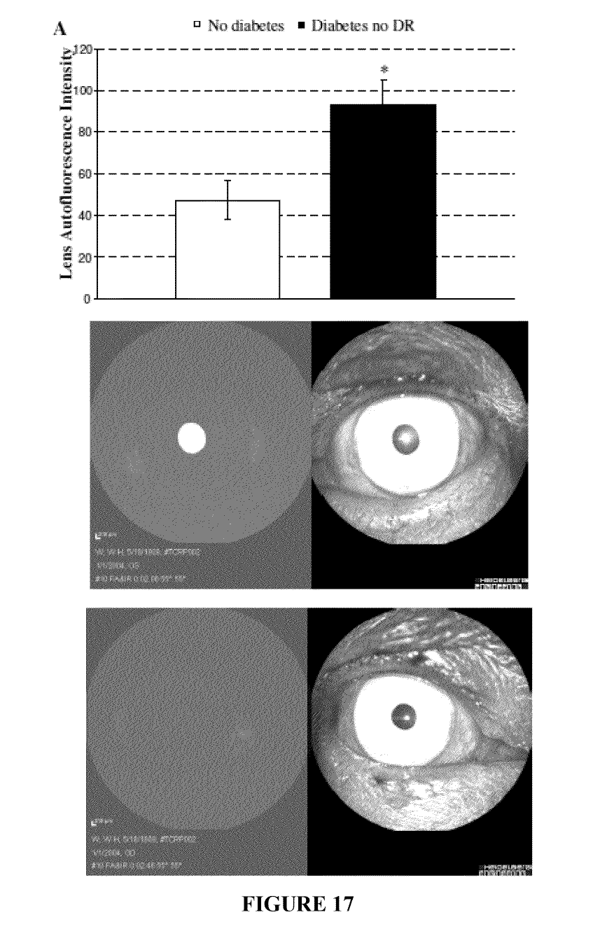

FIG. 17. Scanning laser ophthalmoscope for measuring lens autofluorescence. (A) The average lens autofluorescence for the non-diabetic case is significantly lower than the diabetic with no DR case (p<0.05) where n=3. The data is not gender- or age-matched. (B)-(C). Dual mode images of the left (B) and right (C) eye of the same patient. Autofluorescence observed in B originates from the lens since the axial focus is directed at the anterior eye as confirmed in the IR mode. Autofluorescence is absent in C since the patient's lens has been replaced with an artificial lens due to cataract.

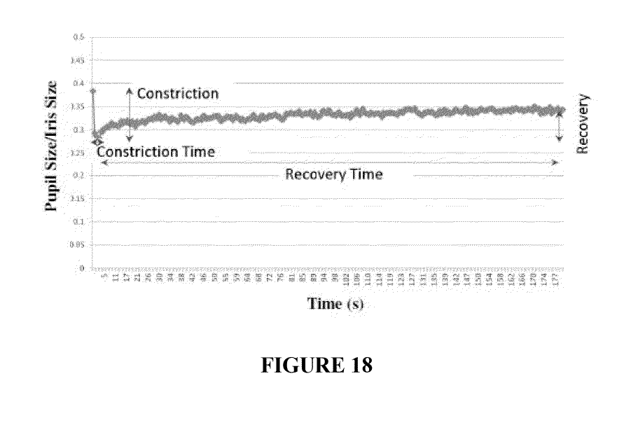

FIG. 18. Pupillary constriction profile for a mydriated eye under sustained exposure to blue laser light. Despite mydriasis, sustained exposure to blue light results in constriction followed by re-dilation (recovery) of the pupil due to light adaptation. The y-axis represents the normalized pupil size which is defined here as the ratio of pupil size to iris size. The basis for using this measure rather than the absolute pupil size is illustrated in FIG. 16.

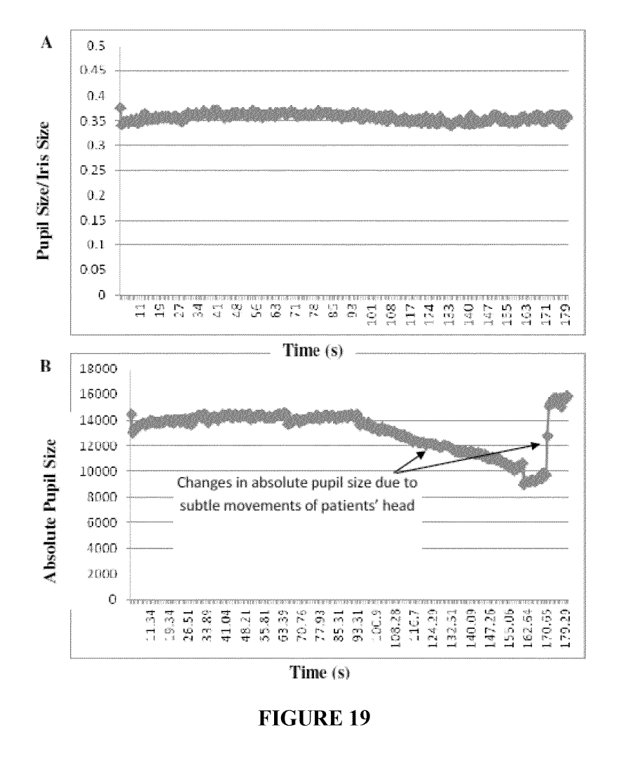

FIG. 19. Comparison between the normalized pupil size/iris size (A) and the absolute pupil size (B). As observed, the pupil constriction profile in B is sensitive to subtle movements of patients' head despite the use of a chin rest and a head rest. In contrast, the normalized profile in A is robust against such variations.

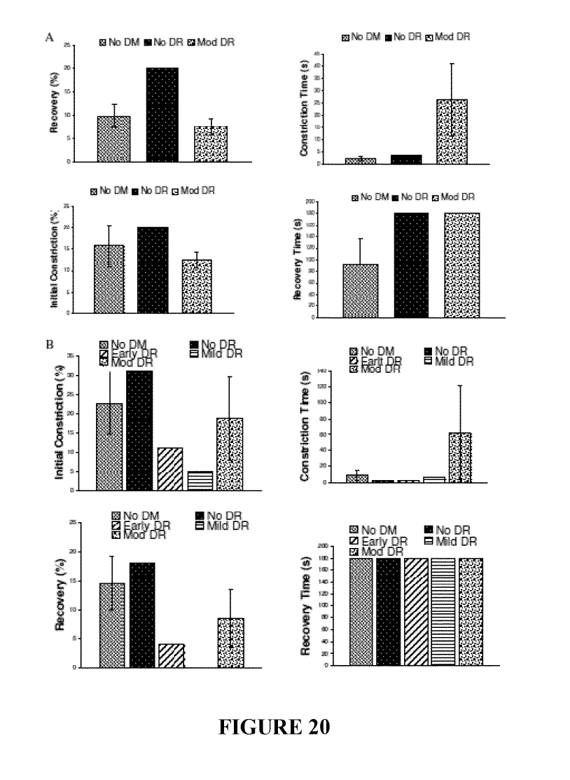

FIG. 20. The quantification of PLR measurements to discriminate between the various stages of DR from mydriated eyes. A. OS (left eye) B. OD (right eye). Patients (i) without diabetes (DM) and (ii) with DM but no DR show, on average, higher initial constriction than patients with early/moderate DR. Pupil constriction time for moderate DR is, on average, longer than (i) patients with no DM (ii) DM with no DR and (iii) DM with early DR. Patients with early/moderate DR have, on average, poorer recovery than patients with (i) no DM and (ii) DM with no DR. All subjects tolerated the procedure and no side effects were reported.

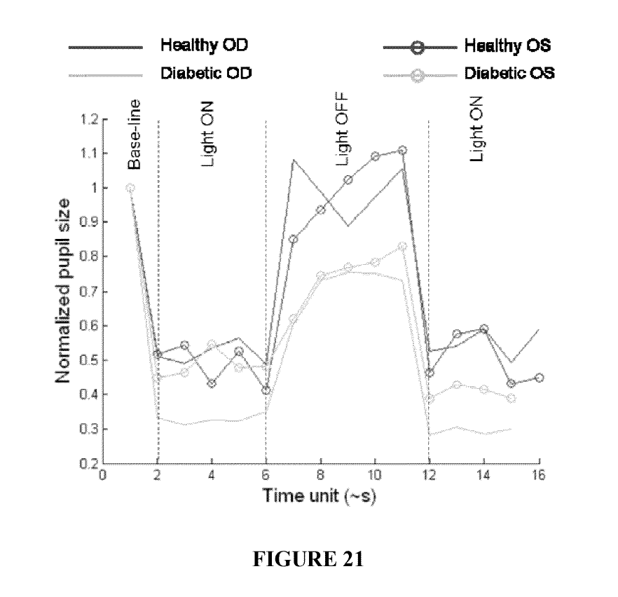

FIG. 21. The quantification of PLR measurements from non-mydriated eyes. A total of 2 subjects volunteered for this study (1: negative control, 1: diabetic but no DR). The normalized pupil size is quantified at fixed time intervals throughout the 3 minute pulsing of light. For every pulsing cycle (once every five seconds or 0.2 Hz), the degree of pupillary constriction and constriction time during light ‘ON’ and degree of pupillary redilation and redilation time during light ‘OFF’ are recorded. As observed, the degree of constriction appears to be more significant in the diabetic patient but the extent of recovery is more significant in the healthy patient. This data suggests potential differences between diabetic and healthy eyes.

Results

The aim here is to demonstrate (i) the robustness of the pupillary light reflex measurement under partial occlusion from eye lid and/or eye lashes as well as due to subtle movements of the patients' head (ii) the efficacy of this approach in characterizing differences between diabetic but non-diabetic retinopathy (DR) and patients with early DR.

Robust Pupil Segmentation Under Partial Occlusion from Eye Lashes During Light “ON” Phase.

FIG. 15 shows that the proposed approach accurately segments the pupil region during light “ON” phase despite partial occlusion from eye lashes. The robustness is attributed to the empirical selection of structuring elements with the appropriate shape (disk-shaped) and size (radius of 3 and 17 for the binary opening and closing operations respectively). The smaller structuring element ensures that the background noise in FIG. 15B is minimized (as observed in FIG. 15C) whereas the larger structuring element ensures that non-autofluorescing regions in the pupil, due to occlusion from eye lashes, are “closed” together so that the complete pupil region is segmented.

Robust Pupil and Iris Segmentation Under Partial Occlusion from Eye Lid and Eye Lashes During Light “OFF” Phase.

FIG. 16 shows that the proposed approach accurately segments the iris and pupil regions during the light “OFF” phase despite partial occlusion from eye lashes. The results follow from the iris and pupil segmentation steps illustrated in FIGS. 11, 13 and 14.

Measuring Lens Autofluorescence.

FIG. 17 demonstrates the use of the dual mode imaging approach for measuring lens autofluorescence where lens autofluorescence data is used here to distinguish diabetic patients with no DR from the negative control. The average lens autofluorescence for the non-diabetic case is significantly lower than the diabetic with no DR case (p<0.05) where n=3 and the data is not gender- or age-matched. Autofluorescence observed in FIG. 17B originates from the lens since the axial focus is directed at the anterior eye as confirmed in the IR mode. Autofluorescence is absent in Figure FIG. 17C since the patient's lens has been replaced with an artificial lens due to cataract.

Normalized Pupil Size Removes Inaccuracies Due to Subtle Movements of Patients' Heads.

FIG. 18 shows a typical pupil constriction profile of a mydriated eye under sustained exposure to blue laser light. Pupil constriction is observed despite mydriasis followed by re-dilation (recovery) of the pupil due to light adaptation. FIG. 19 illustrates the need for using a normalized pupil size to remove inaccuracies due to subtle movements in patients' head (19A). A forward or backward tilt of the patients' head results in an apparent enlargement or reduction in pupil size as perceived by the optical detection system (19B). This artifact can be removed by normalizing the pupil size with respect to the iris size as shown in FIG. 19A since both the pupil and iris would be subject to the same enlargement or reduction in size.

Characterizing Differences Between DR and Non-DR Patients Under Mydriasis.

FIG. 20 characterizes the differences between DR and non-DR patients under mydriasis for both the left (20A) and right (20B) eyes. Patients (i) without diabetes (DM) and (ii) with DM but no DR show, on average, higher initial constriction than patients with early/moderate DR. Pupil constriction time for moderate DR is, on average, longer than (i) patients with no DM (ii) DM with no DR and (iii) DM with early DR. Patients with early/moderate DR have, on average, poorer recovery than patients with (i) no DM and (ii) DM with no DR. All subjects tolerated the procedure and no side effects were reported.

Characterizing Differences Between Non-Mydriated Negative Control and “Diabetic with Non-DR” Patients.

FIG. 21 characterizes differences between non-mydriated negative control and “diabetic with non-DR” patients. As observed, the degree of constriction appears to be more significant in the diabetic patient but the extent of recovery is more significant in the healthy patient. Preliminary data suggests potential differences between diabetic and healthy eyes which, hopefully, clinical validation on a large cohort of patients (>200) would help to establish.

### Example 3

62 diabetic patients were screened using the dual mode method.

Methods.

Patient eyes were subjected to infrared light over a total 3 minute period. Pupil IR image was captured every 1 second over the 3 minutes. Simultaneously, for the 3 minute period, the eyes were subjected to blue light, which was repeatedly turned on for 5 seconds, off for 5 seconds. The pupil region was segmented as described in Example 2 for each IR data frame captured and the pupil size (in pixels) was calculated. For each light ON/OFF cycle, the ratio of pupil maxima/pupil minima was computed, where pupil maxima denotes the largest pupil size during light OFF and pupil minima denotes smallest pupil size during light ON. The average ratio was computed for data combined from all ON/OFF cycles within the 3 minute period.

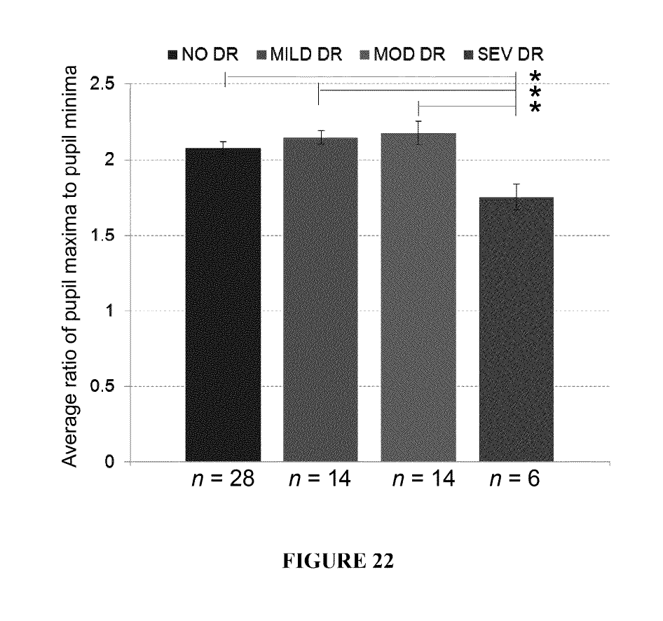

FIG. 22.

Bar chart depicting the average ratio of pupil maxima to pupil minima; from left to right: NO DR (patients with diabetes but no diabetic retinopathy), MILD DR (patients with diabetes and mild diabetic retinopathy), MOD DR (patients with diabetes and moderate diabetic retinopathy), SEV DR (patients with diabetes and severe diabetic retinopathy).

Results.

The results are shown in FIG. 22, in which the y-axis of the bar chart indicates the average ratio calculated. A significant influence was seen due to gender (based on 4-way ANOVA). The group with severe diabetic retinopathy was markedly different from the other 3 groups. It should be noted that the patient populations did not contain a control group that had neither diabetes nor retinopathy. The “No DR” group refers to patients with diabetes but not yet diabetic retinopathy. It may be that even the NO DR group already has impaired pupillary function, which may be absent in a NO DB, NO DR group.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

As used in this specification and the appended claims, the singular forms “a”, “an” and “the” include plural reference unless the context clearly dictates otherwise. As used in this specification and the appended claims, the terms “comprise”, “comprising”, “comprises” and other forms of these terms are intended in the non-limiting inclusive sense, that is, to include particular recited elements or components without excluding any other element or component. As used in this specification and the appended claims, all ranges or lists as given are intended to convey any intermediate value or range or any sublist contained therein. Unless defined otherwise all technical and scientific terms used herein have the same meaning as commonly understood to one of ordinary skill in the art to which this invention belongs.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### REFERENCES

Aiello L P, Gardner T W, King G L, Blankenship G, Cavallerano J D, Ferris F L, Klein R: Diabetic retinopathy. Diabetes Care 21:143-156, 1998
Saine P J: Landmarks in the historical development of fluorescein angiography. Journal of Ophthalmic Photography 15:17-23, 1993
Antonetti D A, Barber A J, Bronson S K, Freeman W M, Gardner T W, Jefferson L S, Kester M, Kimball S R, Krady J K, Lalloue K F, Norbury C C, Quinn P G, Sandirasegarane L, Simpson I A, Grp JDRC: Diabetic retinopathy—Seeing beyond glucose-induced microvascular disease. Diabetes 55:2401-2411, 2006
Schwartz S G, Flynn H W: Pharmacotherapies for Diabetic Retinopathy: Present and Future. Experimental Diabetes Research, 2007
Lieth E, Gardner T W, Barber A J, Antonetti D A, Penn State Retina Res G: Retinal neurodegeneration: early pathology in diabetes. Clinical and Experimental Ophthalmology 28:3-8, 2000
Lorenzi M, Gerhardinger C: Early cellular and molecular changes induced by diabetes in the retina. Diabetologia 44:791-804, 2001
Gardner T W, Antonetti D A, Barber A J, Lalloue K F, Levison S W, Penn State Retina Res G: Diabetic retinopathy: More than meets the eye. Survey of Ophthalmology 47:S253-S262, 2002
Smith S B: The impact of diabetes on neuronal, glial, and vascular cells of the retina. In Retinal Degenerations, Humana Press, 2007, p. 165-183
Serrarbassa P D, Dias A F, Vieira M F: New concepts on diabetic retinopathy: neural versus vascular damage. Arq Bras Oftalmol 71:459-463, 2008
Lucas R J, Douglas R H, Foster R G: Characterization of an ocular photopigment capable of driving pupillary constriction in mice. Nature Neuroscience 4:621-626, 2001
Hattar S, Liao H W, Takao M, Berson D M, Yau K W: Melanopsin-containing retinal. ganglion cells: Architecture, projections, and intrinsic photosensitivity. Science 295:1065-1070, 2002
Tu D C, Zhang D Y, Demas J, Slutsky E B, Provencio I, Holy T E, Van Gelder R N: Physiologic diversity and development of intrinsically photosensitive retinal ganglion cells. Neuron 48:987-999, 2005
Wong K Y, Dunn F A, Berson D M: Photoreceptor adaptation in intrinsically photosensitive retinal ganglion cells. Neuron 48:1001-1010, 2005
Schmidt T M, Kofuji P: Functional and Morphological Differences among Intrinsically Photosensitive Retinal Ganglion Cells. Journal of Neuroscience 29:476-482, 2009
Belenky M A, Smeraski C A, Provencio I, Sollars P J, Pickard G E: Melanopsin retinal ganglion cells receive bipolar and amacrine cell synapses. Journal of Comparative Neurology 460:380-393, 2003
Hattar S, Lucas R J, Mrosovsky N, Thompson S, Douglas R H, Hankins M W, Lem J, Biel M, Hofmann F, Foster R G, Yau K W: Melanopsin and rod-cone photoreceptive systems account for all major accessory visual functions in mice. Nature 424:76-81, 2003
Lucas R J, Hattar S, Takao M, Berson D M, Foster R G, Yau K W: Diminished pupillary light reflex at high irradiances in melanopsin-knockout mice. Science 299:245-247, 2003
Fu Y B, Zhong H N, Wang M H H, Luo D G, Liao H W, Maeda H, Hattar S, Frishman U, Yau K W: Intrinsically photosensitive retinal ganglion cells detect light with a vitamin A-based photopigment, melanopsin. Proceedings of the National Academy of Sciences of the United States of America 102:10339-10344, 2005
Sekaran S, Lupi D, Jones S L, Sheely C J, Hattar S, Yau K W, Lucas R J, Foster R G, Hankins M W: Melanopsin-dependent photoreception provides earliest light detection in the mammalian retina. Current Biology 15:1099-1107, 2005
Barnard A R, Hattar S, Hankins M W, Lucas R J: Melanopsin regulates visual processing in the mouse retina. Current Biology 16:389-395, 2006
Guler A D, Ecker J L, Lall G S, Haq S, Altimus C M, Liao H W, Barnard A R, Cahill H, Badea T C, Zhao H Q, Hankins M W, Berson D M, Lucas R J, Yau K W, Hattar S: Melanopsin cells are the principal conduits for rod-cone input to non-image-forming vision. Nature 453:102-+, 2008
Hankins M W, Peirson S N, Foster R G: Melanopsin: an exciting photopigment. Trends in Neurosciences 31:27-36, 2008
Grozdanic S D, Matic M, Sakaguchi D S, Kardon R H: Evaluation of retinal status using chromatic pupil light reflex activity in healthy and diseased canine eyes. Investigative Ophthalmology & Visual Science 48:5178-5183, 2007
Wang H Z, Lu Q J, Wang N L, Liu H, Zhang L, Zhan G L: Loss of melanopsin-containing retinal ganglion cells in a rat glaucoma model. Chinese Medical Journal 121:1015-1019, 2008
Gastinger M J, Kunselman A R, Conboy E E, Bronson S K, Barber A J: Dendrite remodeling and other abnormalities in the retinal ganglion cells of Ins2(Akita) diabetic mice. Investigative Ophthalmology & Visual Science 49:2635-2642, 2008
Maass A, von Leithner P L, Luong V, Guo L, Salt T E, Fitzke F W, Cordeiro M F: Assessment of rat and mouse RGC apoptosis imaging in vivo with different scanning laser ophthalmoscopes. Curr Eye Res 32:851-861, 2007
Leung C K, Lindsey J D, Crowston J G, Ju W K, Liu Q, Bartsch D U, Weinreb R N: In vivo imaging of murine retinal ganglion cells. J Neurosci Methods 168:475-478, 2008
Hreidarsson A B: Pupil size in insulin-dependent diabetes. Relationship to duration, metabolic control, and long-term manifestations. Diabetes 31:442-448, 1982
Devos A, Marcus J T, Reulen J P H, Peters H F M, Heimans J J, Vanderveen E A: The pupillary light reflex in diabetes-mellitus—Evaluation of a newly developed infrared light-reflection method. Diabetes Research Clinical and Experimental 10:191-195, 1989
Kuroda N, Taniguchi H, Baba S, Yamamoto M: Pupillary light reflex in borderline diabetes-mellitus. Diabetes Research and Clinical Practice 6:89-94, 1989
Fountas K N, Kapsalaki E Z, Machinis T G, Boev A N, Robinson J S, Troup E C: Clinical implications of quantitative infrared pupillometry in neurosurgical patients. Neurocritical Care 5:55-60, 2006
Lin B, Koizumi A, Tanaka N, Panda S, Masland R H: Restoration of visual function in retinal degeneration mice by ectopic expression of melanopsin. Proceedings of the National Academy of Sciences of the United States of America 105:16009-16014, 2008
Berson D M: Phototransduction in ganglion-cell photoreceptors. Pflugers Archiv—European Journal of Physiology 454:849-855, 2007
Bos J E: Clinical quantitative pupillometry—The latency of pupillary constriction as measured with the iris system (preliminary-results). Neuro-Ophthalmology 8:299-305, 1988
Do M T H, Kang S H, Xue T, Zhong H N, Liao H W, Bergles D E, Yau K W: Photon capture and signalling by melanopsin retinal ganglion cells. Nature 457:281-U282, 2009
Martin P M, Roon P, Van Ells T K, Ganapathy V, Smith S B: Death of retinal neurons in streptozotocin-induced diabetic mice. Investigative Ophthalmology & Visual Science 45:3330-3336, 2004
Sholl D A: Dendritic organization in the neurons of the visual and motor cortices of the cat. Journal of Anatomy 87:387-406, 1953

1. A method of monitoring retinopathy in a subject, the method comprising:
directing pulses of high intensity blue light at the retina of an eye of the subject subject over a total time period;
directing high intensity infra-red light at the anterior region of the eye of the subject over the total time period;
measuring autofluorescence of the retina in response to the blue light to obtain an autofluorescence intensity profile;
measuring infra-red reflectance of the anterior region of the eye in response to the infra-red light to obtain an infra-red reflectance intensity profile;
processing the autofluorescence intensity profile and the infra-red reflectance profile to obtain a pupillary light reflex measurement in order to assess the retinopathy status of the retina.

2. The method of claim 1, wherein the blue light has a wavelength of from about 485 nm to about 490 nm.

3. The method of claim 1, wherein the blue light has a wavelength of about 488 nm.

4. The method of claim 1, wherein the infra-red light has a wavelength of from about 800 nm to about 850 nm.

5. The method of claim 1, wherein the infra-red light has a wavelength of about 820 nm.

6. The method of claim 1, wherein a confocal light source is used to produce the blue light and the infra-red light.

7. The method of claim 1, wherein a laser light source is used to produce the blue light and the infra-red light.

8. The method of claim 6, wherein the confocal light source is provided by a confocal scanning laser ophthalmoscope.

9. The method of claim 1, wherein the processing comprises identifying a constricted pupil area from the infra-red reflectance intensity profile and identifying a dilated pupil area from the infra-red reflectance profile.

10. The method of claim 1, wherein processing further comprises obtaining a lens autofluorescence measurement from the autofluorescence intensity profile.

11. The method of claim 1, wherein assessing comprises comparing the processed pupillary light reflex measurement with a processed pupillary light reflex measurement obtained for an eye of a non-diseased individual.

12. The method of claim 10, wherein assessing comprises comparing the processed lens autofluorescence measurement with a processed lens autofluorescence measurement obtained for an eye of a non-diseased individual.

13. The method of claim 1, wherein assessing comprises comparing the processed pupillary light reflex measurement with a processed the processed pupillary light reflex measurement obtained for the same eye of the subject.

14. The method of claim 10, wherein assessing comprises comparing the processed lens autofluorescence measurement with a processed lens autofluorescence measurement obtained for the same eye of the subject.

15. A diagnostic tool for monitoring retinopathy, the diagnostic tool comprising:
a light source for generating high intensity blue light and high intensity infra-red light;
a detector for detecting autofluorescence of a retina in response to the blue light and infra-red reflectance of an anterior region of an eye in response to the infra-red light;
a memory, said memory storing instructions; and
a processor in communication with said light source, said detector and said memory, said processor executing instructions to:
activate said light to generate pulses of said high intensity blue light directed at the retina of an eye of a subject;
activate said light source to generate said high intensity infra-red light directed at the anterior region of the eye of the subject;
obtain an autofluorescence profile over a total time period and an infra-red reflectance profile over the total time period from measurements at said detector; and
process said autofluorescence intensity profile and said infra-red reflectance profile to obtain a pupillary light reflex measurement to assess the retinopathy status of the retina.

16. The diagnostic tool of claim 15, wherein said light source is a confocal light source.

17. The diagnostic tool of claim 15, wherein said light source is a laser light source.

18. The diagnostic tool of claim 15, wherein said light source is a confocal scanning laser.

19. The diagnostic tool of claim 18, wherein said confocal scanning laser produces light at a wavelength of about 485 nm to about 490 nm and/or a wavelength of about 800 nm to about 850 nm.

20-22. (canceled)

23. A computer-readable medium storing executable instructions that, upon execution by a processor of a computing device, causes said computing device to facilitate monitoring of retinopathy by:
generating pulses of high intensity blue light for directing at the retina of an eye of a subject and generating high intensity infra-red light for directing at the anterior region of the eye of the subject;
measuring autofluorescence of the retina in response to the blue light over a total time period to obtain an autofluorescence profile;
measuring infra-red reflectance of the anterior region of the eye in response to the infra-red light over a total time period to obtain an infra-red reflectance profile; and
processing the autofluorescence intensity profile and the infra-red reflectance profile to obtain a pupillary light reflex measurement to assess the retinopathy status of the retina.
